(19) United States
(12) Patent Application Publication
(10) Pub. No.: US 2021/0338672 A1
(43) Pub. Date: Nov. 4, 2021

(54) USE OF AN INHIBITOR OF AN ENT FAMILY TRANSPORTER IN THE TREATMENT OF CANCER AND COMBINATION THEREOF WITH AN ADENOSINE RECEPTOR ANTAGONIST

(71) Applicant: iTeos Belgium SA, Gosselies (BE)

(72) Inventors: Stefano CROSIGNANI, Nivelles (BE); Erica Joke Katelijne Heleen HOUTHUYS, Petit-Enghien (BE); Oliver DE HENAU, Watermaele-Boitsfort (BE)

(21) Appl. No.: 17/281,156

(22) PCT Filed: Sep. 27, 2019
(86) PCT No.: PCT/EP2019/076244

§ 371 Date: Mar. 29, 2021

Related U.S. Application Data
(60) Provisional application No. 62/737,717, filed on Sep. 27, 2018.

(30) Foreign Application Priority Data
Sep. 27, 2018 (BE) 2018/0115

(51) Int. Cl. A61K 31/506; A61K 35/28; A61P 35/00; A61K 31/519; A61K 31/5377; A61K 31/541; A61K 31/53

(52) U.S. Cl. CPC A61K 31/506; A61K 35/28; A61P 35/00; A61K 31/53; A61K 31/5377; A61K 31/541; A61K 31/519

(57) ABSTRACT

The present invention relates to the use of an inhibitor of an ENT family transporter for the treatment of cancer. The invention further relates to the combined use of such inhibitor of an ENT family transporter with an adenosine receptor antagonist, for the treatment of cancer. The invention further relates to a pharmaceutical composition and a kit of parts comprising such combination.

### FIELD OF INVENTION

The present invention relates to the use of an inhibitor of an ENT family transporter for the treatment of cancer. The invention further relates to the combined use of such inhibitor of an ENT family transporter with an adenosine receptor antagonist, for the treatment of cancer. The invention further relates to a pharmaceutical composition and a kit of parts comprising such combination.

### BACKGROUND OF INVENTION

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfil important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

A variety of drugs such as dilazep, dipyridamole, and draflazine interact with ENTs and alter adenosine levels, and were developed for their cardioprotective or vasodilatory effects.

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920) (Stagg and Smyth, Oncogene, 2010, 2, 5346-5358) (Antonioli L et al., Nature Reviews Cancer, 2013, 13, 842-857) (Cekic C and Linden J, Nature Reviews, Immunology, 2016, 16, 177-192) (Allard B et al., Curr Op Pharmacol, 2016, 29, 7-16) (Vijayan D et al., Nature Reviews Cancer, 2017, 17, 709-724).

To assess whether adenosine might reduce T cell vitality through intracellular uptake by ENTs, mRNA expression levels of plasma membrane-localized ENT transporters (ENT1, ENT2 and ENT4) in human primary lymphocytes was checked in a publicly available RNA-seq database (Bonnal R J P et al., Nature, 2015, 2:150051). B cells, CD4⁺ and CD8⁺ T cells expressed ENT1 (SLC29A1), ENT2 (SLC29A2) and ENT4 (SLC29A4) (FIGS. 1A, 1B and 1C).

The Applicant herein shows that adenosine as well as ATP profoundly suppress T cell proliferation and cytokine secretion (IL-2), and strongly reduce T cell viability. Adenosine- and ATP-mediated suppression of T cell viability and proliferation were successfully restored using ENTs inhibitors.

Moreover, the use of an ENT inhibitor in combination with an adenosine receptor antagonist enabled to restore not only adenosine- and ATP-mediated suppression of T cell viability and proliferation, but also restored T cell cytokine secretion.

Therefore, the present invention provides the use of an inhibitor of an ENT family transporter for the treatment of cancer. It also provides the combined use of such inhibitor of an ENT family transporter with an adenosine receptor antagonist, for the treatment of cancer.

### SUMMARY

This invention thus relates to a method of treating cancer, comprising: administering, to a human subject in need thereof, an effective amount of an inhibitor of an ENT family transporter.

In one embodiment, the ENT family transporter is ENT1, and the inhibitor is selected from the group consisting of a small molecule, a nucleic acid, a peptide, and an antibody.

In one embodiment, the subject is treated with an additional therapeutic agent in combination with the inhibitor of the ENT family transporter, or has received the additional therapeutic agent within about fourteen days of administration of the inhibitor of the ENT family transporter. In one embodiment, the additional therapeutic agent comprises an adenosine receptor antagonist.

In one embodiment, the subject has previously received at least one prior therapeutic treatment, and has progressed subsequent to the administration of the at least one prior therapeutic treatment and prior to administration of the inhibitor of an ENT family transporter. In one embodiment, the prior therapeutic treatment is selected from the group consisting of chemotherapy, immunotherapy, radiation therapy, stem cell transplant, hormone therapy, and surgery.

The invention also provides a dosage formulation, comprising: an ENT family transporter inhibitor in an amount effective to treat cancer in a human subject.

In one embodiment, the ENT family transporter inhibitor is administered prior to, concomitant with, or subsequent to administration of an additional therapeutic agent comprising an adenosine receptor antagonist.

The invention also relates to a method of treating cancer, comprising: administering, to a patient in need thereof, a combination of an adenosine receptor antagonist and an inhibitor of an ENT family transporter.

In one embodiment, the adenosine receptor antagonist is an A2A or A2B receptor antagonist.

In one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine; 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, the adenosine receptor antagonist is a compound of Formula (I), as defined hereafter.

In one embodiment, the ENT family member is ENT1.

In one embodiment, the A2A or A2B receptor antagonist and the ENT1 inhibitor are provided in the same formulation.

The invention further provides a formulation, comprising: an effective amount of an adenosine receptor antagonist in combination with an effective amount of an inhibitor of an ENT family member, along with a pharmaceutically acceptable excipient.

In one embodiment, in the formulation, the adenosine receptor antagonist is an A2A or A2B receptor antagonist.

In one embodiment, in the formulation, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, in the formulation, the adenosine receptor is a compound of Formula (I) as defined hereafter.

In one embodiment, in the formulation, the ENT family member inhibitor is an ENT1 inhibitor.

In one embodiment, the formulation further comprises an additional therapeutic agent.

### Definitions

In the present invention, the following terms have the following meanings:

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “alkyl” refers to a hydrocarbyl radical of formula CₙH₂ₙ₊₁ wherein n is a number greater than or equal to 1. Generally, alkyl groups of this invention comprise from 1 to 8 carbon atoms, more preferably, alkyl groups of this invention comprise from 1 to 6 carbon atoms. Alkyl groups may be linear or branched. Suitable alkyl groups include methyl, ethyl, propyl, butyl, pentyl, hexyl, heptyl and octyl.

The term “alkylaminoalkyl” refers to a group -alkyl-NH-alkyl wherein alkyl is as herein defined.

The term “alkylaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylheteroaryl” refers to any heteroaryl substituted by an alkyl group wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkyne” refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 5 to 10; more preferably the aryl is a 5- or 6-membered aryl. Non-limiting examples of aryl comprise phenyl, naphthalenyl.

The term “carbonyl” refers to a group —(C═O)—.

The term “carbonylamino” refers to a group —NH—(C═O)—.

The term “cycloalkyl” refers to a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms; still more preferably more preferably the cycloalkyl is a 5- or 6-membered cycloalkyl. Examples of cycloalkyl groups include but are not limited to cyclopropyl, cyclobutyl, cyclopentyl, cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bromo, or iodo.

The term “heteroaryl” refers to an aryl group as herein defined wherein at least one carbon atom is replaced with a heteroatom. In other words, it refers to 5 to 12 carbon-atom aromatic single rings or ring systems containing 2 rings which are fused together, typically containing 5 to 6 atoms; in which one or more carbon atoms is replaced by oxygen, nitrogen and/or sulfur atoms where the nitrogen and sulfur heteroatoms may optionally be oxidized and the nitrogen heteroatoms may optionally be quaternized. Non-limiting examples of such heteroaryl, include: oxazolyl, thiazolyl, imidazolyl, furanyl and pyrrolyl. Preferably the heteroaryl is a 5- or 6-membered heteroaryl, more preferably the 5- or 6-membered heteroaryl is a furyl.

The term “heterocyclyl” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Preferably the heterocyclyl is a 5- or 6-membered heterocyclyl. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, 1-oxido-1-thiomorpholin-4-yl, 1-dioxido-1-thiomorpholin-4-yl, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “heterocyclylalkylaminocarbonyl” refers to a group —(C═O)—NH-alkyl-heterocyclyl, wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “heterocyclylcarbonyl” refers to a group —(C═O)-heterocyclyl wherein heterocyclyl is as herein defined.

The term “heterocyclylalkyl” refers to a group -alkyl-heterocyclyl wherein alkyl and heterocyclyl are as herein defined.

The term “heterocyclyloxy” to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined. In other words, “hydroxycarbonyl” corresponds to a carboxylic acid group.

The term “oxo” refers to a ═O substituent.

The term “sulfonylamino” refers to a group —NH—SO₂.

The term “about”, preceding a figure encompasses plus or minus 10%, or less, of the value of said figure. It is to be understood that the value to which the term “about” refers is itself also specifically, and preferably, disclosed.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “antagonist” refers to a natural or synthetic compound which binds to the protein and blocks the biological activation of the protein, and thereby the action of the said protein. The protein may be a receptor, i.e. a protein molecule that receives chemical signals from outside a cell. Consequently, “an adenosine receptor antagonist” includes any chemical entity that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein. Consequently, an “ENT inhibitor” or “inhibitor of an ENT family transporter” refers to a compound that has a biological effect to inhibit or significantly reduce or down-regulate the biological activity of ENT family transporter.

The term “antibody” as used herein includes monoclonal antibodies, polyclonal antibodies, multispecific antibodies (e.g., bispecific antibodies), and antibody fragments, so long as they exhibit the desired biological activity.

The term “chemotherapy” refers to a type of cancer treatment that uses one or more anti-cancer drugs (chemotherapeutic agents) as part of a standardized chemotherapy regimen. Chemotherapy may be given with a curative intent or it may aim to prolong life or to reduce symptoms. Chemotherapeutic agents are for example selected from anticancer alkylating agents, anticancer antimetabolites, anticancer antibiotics, plant-derived anticancer agents, anticancer platinum coordination compounds and any combination thereof.

The term “hormone therapy” refers to the use of hormones in medical treatment. In one embodiment, the hormone therapy is oncologic hormone therapy.

The term “human” refers to a subject of both genders and at any stage of development (i.e. neonate, infant, juvenile, adolescent, adult).

The term “patient” refers to a mammal, more preferably a human, who/which is awaiting the receipt of, or is receiving medical care or is/will be the object of a medical procedure.

The term “immunotherapy” refers to a therapy aiming at inducing and/or enhancing an immune response towards a specific target, for example towards cancer cells. Immunotherapy may involve the use of checkpoint inhibitors, checkpoint agonists (also called T-cell agonists), IDO inhibitors, PI3K inhibitors, adenosine receptor inhibitors, adenosine-producing enzymes inhibitors, adoptive transfer, therapeutic vaccines, and combinations thereof.

The term “nucleic acid” refers to a polymer of nucleotides covalently linked by phosphodiester bonds, such as deoxyribonucleic acids (DNA) or ribonucleic acids (RNA), in either single- or double-stranded form. Unless specifically limited, the term encompasses nucleic acids containing known analogues of natural nucleotides that have similar binding properties as the reference nucleic acid and are metabolized in a manner similar to naturally occurring nucleotides.

The term “peptide” refers to a linear polymer of amino acids of less than 50 amino acids linked together by peptide bonds.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “prodrug” as used herein means the pharmacologically acceptable derivatives of compounds of Formula (I), such as for example esters or amides, whose in vivo biotransformation product generates the biologically active drug. Prodrugs are generally characterized by increased bio-availability and are readily metabolized into biologically active compounds in vivo.

The term “radiation therapy” refers to a method of treatment of cancer employing various radiations such as X-ray, gamma-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT); brachytherapy or sealed source radiation therapy; and systemic radioisotope therapy (RIT) or unsealed source radiotherapy.

The terms “therapeutically effective amount” or “effective amount” or “therapeutically effective dose” refer to the amount or dose of active ingredient that is aimed at, without causing significant negative or adverse side effects to the subject, (1) delaying or preventing the onset of a cancer in the subject; (2) reducing the severity or incidence of a cancer; (3) slowing down or stopping the progression, aggravation, or deterioration of one or more symptoms of a cancer affecting the subject; (4) bringing about ameliorations of the symptoms of a cancer affecting the subject; or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present invention, the subject or mammal shows observable and/or measurable reduction in or absence of one or more of the following: reduction of the number of cancer cells; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “stem cell transplant” refers to a procedure in which a patient receives healthy blood-forming cells (stem cells) to replace their own that have been destroyed by disease or by the radiation or high doses of anticancer drugs that are given as part of the procedure. The healthy stem cells may come from the blood or bone marrow of the patient, from a donor, or from the umbilical cord blood of a newborn baby. A stem cell transplant may be autologous (using a patient's own stem cells that were collected and saved before treatment), allogeneic (using stem cells donated by someone who is not an identical twin), or syngeneic (using stem cells donated by an identical twin).

The term “subject” refers to a mammal, preferably a human. In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

### DETAILED DESCRIPTION

### ENT Inhibitor for Use in Treating Cancer

The invention relates to the use of an inhibitor of ENT family transporter for treating cancer.

Hereafter, inhibitors of an ENT family transporter are also referred to as ENT inhibitors.

The equilibrative nucleoside transporter (ENT) family, also known as SLC29, is a group of plasmalemmal transport proteins which transport nucleoside substrates like adenosine into cells. There are four known ENTs, designated ENT1, ENT2, ENT3, and ENT4.

In one embodiment, the ENT inhibitor is an inhibitor of ENT1.

In one embodiment, the ENT inhibitor is an inhibitor which is selective of ENT1. In one embodiment, the ENT inhibitor is an inhibitor which is selective of ENT1 with respect to other ENTs inhibitors, especially with respect to ENT2 and ENT4.

In one embodiment, the ENT inhibitor is selected from the group consisting of a small molecule, a nucleic acid, a peptide, and an antibody.

Examples of ENT inhibitors include dilazep, dipyridamole, NBMPR (nitrobenzylthioinosine), draflazine, STI-571 (Gleevec), ticagrelor, soluflazine, mioflazine, decynium-22, lopinavir, quinidine, 8MDP, TC-T 6000, 5-iodotubercidin, cilostazol, salts thereof and any mixture thereof. In a specific embodiment, the ENT inhibitor is selected from NBMPR, dipyridamole, dilazep, ticagrelor and salts thereof (including dilazep hydrochloride). In a specific embodiment, the ENT inhibitor is selected from dipyridamole, dilazep, ticagrelor and salts thereof (including dilazep hydrochloride). In one embodiment, the ENT inhibitor is NBMP or a salt thereof. In one embodiment, the ENT inhibitor is dipyridamole or a salt thereof. In one embodiment, the ENT inhibitor is dilazep or a salt thereof (including dilazep hydrochloride). In one embodiment, the ENT inhibitor is ticagrelor or a salt thereof.

Examples of ENT1 inhibitors include dilazep, dipyridamole, NBMPR (nitrobenzylthioinosine), draflazine, STI-571 (Gleevec), ticagrelor, 8MDP, 5-iodotubercidin, cilostazol, salts thereof and any mixture thereof. Examples of selective ENT1 inhibitors include NBMPR, STI-571 (Gleevec), ticagrelor, salts thereof and any mixture thereof.

The invention thus relates to an inhibitor of an ENT family transporter for use in the treatment of cancer in a human subject.

In one embodiment, the subject is treated with an additional therapeutic agent in combination with the inhibitor of the ENT family transporter, or has received the additional therapeutic agent within about fourteen days of administration of the inhibitor of the ENT family transporter. In one embodiment, the additional therapeutic agent comprises an adenosine receptor antagonist. In one embodiment, the ENT family transporter inhibitor is administered prior to, concomitant with, or subsequent to administration of the additional therapeutic agent comprising an adenosine receptor antagonist.

In one embodiment, the subject has previously received at least one prior therapeutic treatment, and has progressed subsequent to the administration of the at least one prior therapeutic treatment and prior to administration of the inhibitor of an ENT family transporter. In one embodiment, the prior therapeutic treatment is selected from the group consisting of chemotherapy, immunotherapy, radiation therapy, stem cell transplant, hormone therapy, and surgery.

The invention thus relates to a method of treating cancer, comprising: administering, to a human subject in need thereof, an effective amount of an inhibitor of an ENT family transporter.

In one embodiment, in the method of the invention, the subject is treated with an additional therapeutic agent in combination with the inhibitor of the ENT family transporter, or has received the additional therapeutic agent within about fourteen days of administration of the inhibitor of the ENT family transporter. In one embodiment, in the method of the invention, the additional therapeutic agent comprises an adenosine receptor antagonist. Further details regarding adenosine receptor antagonist are provided below.

In one embodiment, in the method of the invention, the subject has previously received at least one prior therapeutic treatment, and has progressed subsequent to the administration of the at least one prior therapeutic treatment and prior to administration of the inhibitor of an ENT family transporter. In one embodiment, in the method of the invention, the prior therapeutic treatment is selected from the group consisting of chemotherapy, immunotherapy, radiation therapy, stem cell transplant, hormone therapy, and surgery.

The invention also relates to a dosage formulation, comprising an ENT family transporter inhibitor in an amount effective to treat cancer in a human subject.

In one embodiment, in the formulation of the invention, the ENT family transporter inhibitor is administered prior to, concomitant with, or subsequent to administration of an additional therapeutic agent comprising an adenosine receptor antagonist.

Combined Use: ENT Inhibitor with Adenosine Receptor Antagonist

The invention further relates to the combined use of an ENT inhibitor with an adenosine receptor antagonist.

The invention thus relates to a combination comprising:
(a) an effective amount of an ENT inhibitor, and
(b) an effective amount of an adenosine receptor antagonist.

In the context of the present invention the term “combination” preferably means a combined occurrence of an ENT inhibitor and of an A2AR antagonist. Therefore, the combination of the invention may occur either as one composition, comprising all the components in one and the same mixture (e.g. a pharmaceutical composition), or may occur as a kit of parts, wherein the different components form different parts of such a kit of parts. The administration of the ENT inhibitor and of the A2AR antagonist may occur either simultaneously or timely staggered, with similar or different timing of administration (i.e. similar or different numbers of administration of each component), either at the same site of administration or at different sites of administration, under similar of different dosage form.

The invention further relates to a method of treating cancer, comprising: administering, to a patient in need thereof, a combination of an adenosine receptor antagonist and an ENT inhibitor.

Above embodiments relative to the ENT inhibitors also apply to the combination of the invention. Especially, in one embodiment, in the combination of the invention, the ENT inhibitor is an inhibitor of ENT1.

As a second component, the combination of the invention includes at least one adenosine receptor antagonist.

As defined above, “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

Adenosine receptors (or P1 receptors) are a class of purinergic G protein-coupled receptors with adenosine as endogenous ligand. There are four known types of adenosine receptors in humans: A1, A2A, A2B and A3; each is encoded by a different gene (ADOARA1, ADORA2A, ADORA2B, and ADORA3 respectively).

In one embodiment, the adenosine receptor antagonist is an antagonist of A1 receptor, A2A receptor, A2B receptor, A3 receptor or of a combination thereof.

In one embodiment, the adenosine receptor antagonist is an antagonist of A2A receptor, A2B receptor or of a combination thereof. In one embodiment, the adenosine receptor antagonist is an A2A or A2B receptor antagonist.

In one embodiment, the adenosine receptor antagonist is an antagonist of A2A receptor (A2AR antagonist). In one embodiment, the adenosine receptor antagonist is an antagonist of A2B receptor (A2BR antagonist).

In one embodiment, the adenosine receptor antagonist is an antagonist which is selective of A2A receptor with respect to other adenosine receptors. In one embodiment, the adenosine receptor antagonist is an antagonist which is selective of A2A receptor with respect to A2B receptor.

In one embodiment, the adenosine receptor antagonist is an antagonist which is selective of A2B receptor with respect to other adenosine receptors. In one embodiment, the adenosine receptor antagonist is an antagonist which is selective of A2B receptor with respect to A2A receptor.

In a specific embodiment, the combination of the invention comprises at least one A2A receptor antagonist as herein defined and at least one ENT inhibitor as defined above, such as for example at least one ENT1 inhibitor.

### A2A Receptor Antagonist

In one embodiment, the combination of the invention includes at least one A2AR antagonist.

An “A2AR antagonist” refers to a compound that, upon administration to a patient, results in inhibition or down-regulation of a biological activity associated with activation of A2A receptor in the patient, including any of the downstream biological effects otherwise resulting from the binding to A2A receptor of its natural ligand. Such A2AR antagonists include any agent that can block activation of A2A receptor or any of the downstream biological effects of A2A receptor activation.

Examples of A2AR antagonists include: Preladenant (SCH-420,814), Vipadenant (BIIB-014), Tozadenant (SYK-115), ATL-444, Istradefylline (KW-6002), MSX-3, SCH-58261, SCH-412,348, SCH-442,416, ST-1535, Caffeine, VER-6623, VER-6947, VER-7835, ZM-241,385, theophylline. It also includes A2AR antagonists disclosed in WO2018/178338, WO2011/121418, WO2009/156737, WO2011/095626 or WO2018/136700.

In one embodiment, the A2AR antagonist is a thiocarbamate derivative, especially a thiocarbamate derivative as those disclosed in WO2018/178338. More preferably the A2AR antagonist is a thiocarbamate derivative of formula (I) as described below.

Thus in a specific embodiment, the invention provides a combination comprising:
(a) an ENT inhibitor, and
(b) an A2AR antagonist being a thiocarbamate derivative of Formula (I)

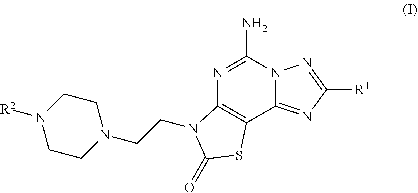

or a pharmaceutically acceptable salt or solvate thereof, wherein R¹ and R² are as defined below.

In a preferred embodiment, the A2AR antagonist is a compound of Formula (I):

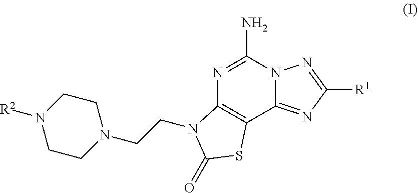

or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
R² represents 6-membered aryl or 6-membered heteroaryl,
   wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
      said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
   or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred compounds of Formula (I) are of Formula (Ia):

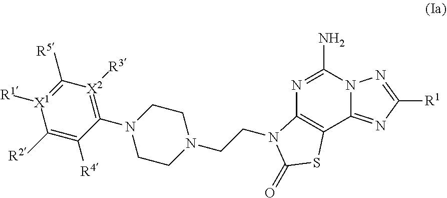

or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{3′} is absent when X² is N; or when X² is C, R^{3′} represents H or halo, preferably H or F;
R^{4′} represents H or halo, preferably H or F; and
R^{5′} represents H or halo, preferably H or F.

In one specific embodiment of the invention, R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro). In a preferred embodiment, R¹ represents 5-membered heteroaryl; more preferably, R¹ represents furyl.

In one specific embodiment of the invention, X¹ and X² represent each independently C or N. In another specific embodiment, X¹ and X² both represent C.

In one specific embodiment of the invention, R^{1′} is absent when X¹ is N.

In another specific embodiment, when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{1′} substituents are optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl.

In one specific embodiment of the invention, R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2′} substituents are optionally substituted by one or more substituent selected from oxo, halo, hydroxy, cyano, alkyl, heterocyclylalkyl, dihydroxyalkyl, dialkylaminoalkyl, heteroaryl, alkylheteroaryl, hydroxycarbonyl, alkyloxycarbonyl, aminocarbonyl, heterocyclylalkylaminocarbonyl, alkylaminoalkylcarbonyl, dialkylaminoalkylcarbonyl, alkylsulfoxide, alkylsulfonealkyl.

In another specific embodiment of the invention, R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{3′} is absent when X² is N. In another specific embodiment of the invention, when X² is C, R^{3′} represents H or halo. In a preferred embodiment, when X² is C, R^{3′} represents H or F.

In one specific embodiment of the invention, R^{4′} represents H or halo. In a preferred embodiment, R^{4′} represents H or F.

In one specific embodiment of the invention, R^{5′} represents H or halo. In a preferred embodiment, R^{5′} represents H or F.

In one embodiment, preferred compounds of Formula (Ia) are those of Formula (Ia-1):

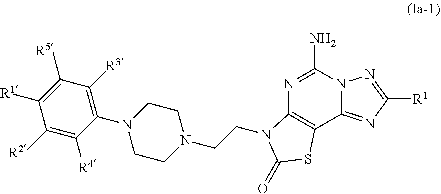

or a pharmaceutically acceptable salt or solvate thereof, wherein R¹, R^{1′}, R^{2′}, R^{3′}, R^{4′} and R^{5′} are as defined in Formula (Ia).

In one embodiment, preferred compounds of Formula (Ia-1) are those of Formula (Ia-1a):

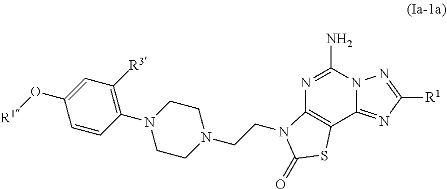

or a pharmaceutically acceptable salt or solvate thereof, wherein:
R¹ and R^{3′} are as defined in Formula (Ia); and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{1″} represents an alkyl or heterocyclyl group substituted by one or more group selected from hydroxy, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide, alkylsulfonealkyl.

In one embodiment, preferred compounds of Formula (Ia-1) are those of Formula (Ia-1b):

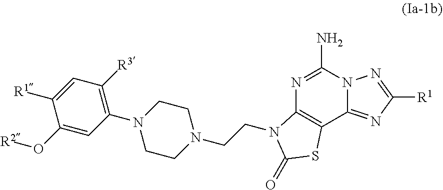

or a pharmaceutically acceptable salt or solvate thereof, wherein:
R¹ and R^{3′} are as defined in Formula (Ia);
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{1′} represents H or halo. In a preferred embodiment, R^{1′} represents H or F.

In one specific embodiment of the invention, R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2″} represents an alkyl or heterocyclyl group substituted by one or more group selected from hydroxy, cyano, heteroaryl, alkylheteroaryl, alkyne, hydroxycarbonyl, alkyloxycarbonyl, aminocarbonyl, alkylaminoalkylcarbonyl, dialkylaminoalkylcarbonyl, alkylsulfoxide, alkylsulfonealkyl.

In one embodiment, preferred compounds of Formula (Ia-1) are those of Formula (Ia-1c) or (Ia-1d):

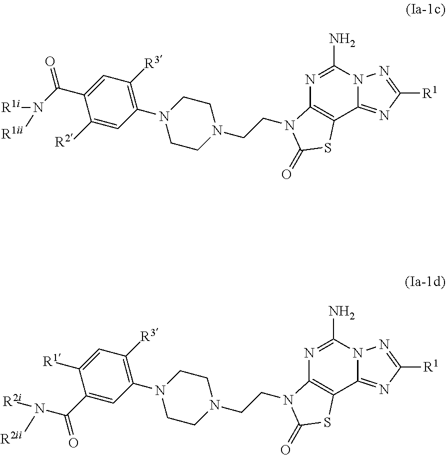

or a pharmaceutically acceptable salt or solvate thereof, wherein:
R¹ and R^{3′} are as defined in Formula (Ia);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In one specific embodiment of the invention, R^{1′} represents H or halo. In a preferred embodiment, R^{1′} represents H or F.

In one specific embodiment of the invention, R^{2′} represents H or halo. In a preferred embodiment, R^{2′} represents H or F.

In one specific embodiment of the invention, R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl. In a preferred embodiment, R²ⁱ and R²ⁱⁱ represent each independently hydrogen, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl or heterocyclylalkylaminocarbonyl.

In one specific embodiment of the invention, R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl. In a preferred embodiment, R²ⁱ and R²ⁱⁱ represent each independently hydrogen, alkyl, heterocyclylalkyl, dihydroxyalkyl, dialkylaminoalkyl or heterocyclylalkylaminocarbonyl. In a preferred embodiment, R²ⁱ and R²ⁱⁱ represent each independently hydrogen, alkyl or dialkylaminoalkyl.

In one embodiment, preferred compounds of Formula (Ia) are those of Formulae (Ia-2) or (Ia-3):

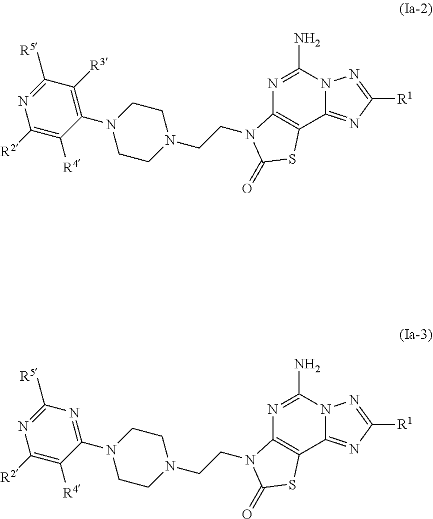

or a pharmaceutically acceptable salt or solvate thereof, wherein R¹, R^{2′}, R^{3′}, R^{4′} and R^{5′} are as defined in Formula (Ia).

Particularly preferred compounds of Formula (I) of the invention are those listed in Table 1 hereafter.

TABLE 1

| Cpd | | | |
|---|---|---|---|
| no | Structure | Chemical name | MW |
| 1 | 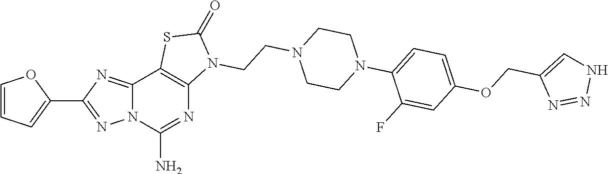 [SMILES: Nc1nc2c(sc(=O)n2CCN2CCN(c3ccc(OCc4c[nH]nn4)cc3F)CC2)c2nc(-c3ccco3)nn12] | 3-(2-(4-(4-((1H-1,2,3- triazolo-4yl)methoxy- 2fluorophenyl)piperazine-1- yl)ethyl)-5-amino-(8-(furan- 2-yl)thiazolo[5,4- e][1,2,4]triazolo[1,5- c]pyrimidine-2(3H)-one | 577.60 |
| 2 | 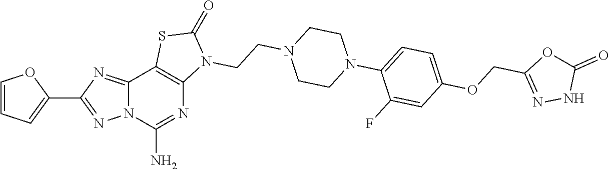 [SMILES: Nc1nc2c(sc(=O)n2CCN2CCN(c3ccc(OCc4n[nH]c(=O)o4)cc3F)CC2)c2nc(-c3ccco3)nn12] | 5-((4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)methyl)- 1,3,4-oxadiazol-2(3H)-one | 594.58 |
| 3 | 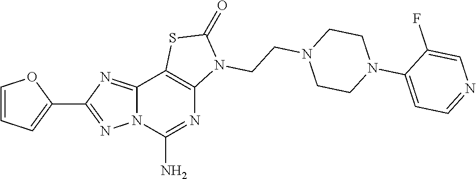 [SMILES: Nc1nc2c(sc(=O)n2CCN2CCN(c3ccncc3F)CC2)c2nc(-c3ccco3)nn12] | 5-amino-3-(2-(4-(3- fluoropyridin-4- yl)piperazin-1-yl)ethyl)-8- (furan-2-yl)thiazolo[5,4- e][1,2,4]triazolo[1,5- c]pyrimidin-2(3H)-one | 481.51 |
| 4 | 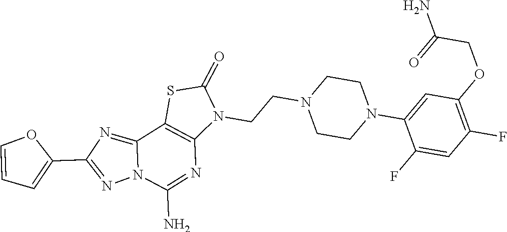 [SMILES: NC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4-e] [1,2,4]triazolo[1,5-c] pyrimidin-3(2H)- yl)ethyl)piperazin-1-yl)-2,4- difluorophenoxy)acetamide | 571.56 |
| 5 | 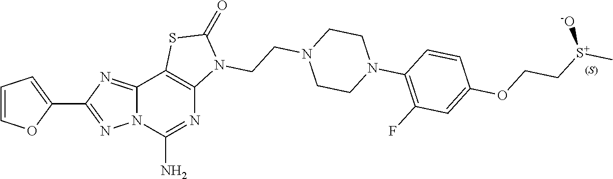 | (S)-5-amino-3-(2-(4-(2- fluoro-4-(2- (methylsulfinyl)ethoxy) phenyl)piperazin-1-yl)ethyl)- 8-(furan-2-yl)thiazolo[5,4- e][1,2,4]triazolo[1,5- c]pyrimidin-2(3H)-one | 586.66 |
| 6 | 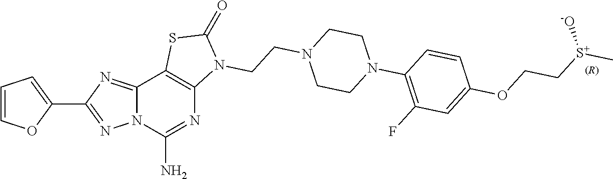 | (R)-5-amino-3-(2-(4-(2- fluoro-4-(2- (methylsulfinyl)ethoxy) phenyl)-piperazin-1-yl)ethyl)- 8-(furan-2-yl)thiazolo[5,4- e][1,2,4]triazolo[1,5- c]pyrimidin-2(3H)-one | 586.66 |
| 7 | 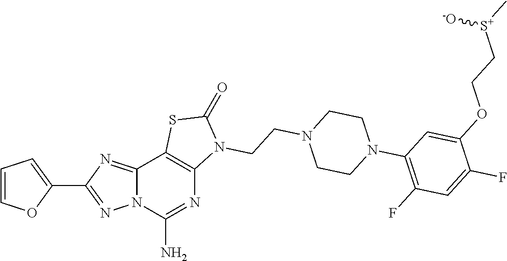 | (R,S)-5-amino-3-(2-(4-(2,4- difluoro-5-(2- (methylsulfinyl)ethoxy) phenyl)piperazin-1-yl)ethyl)- 8-(furan-2-yl)thiazolo[5,4- e][1,2,4]triazolo[1,5- c]pyrimidin-2(3H)-one | 604.65 |
| 8a | 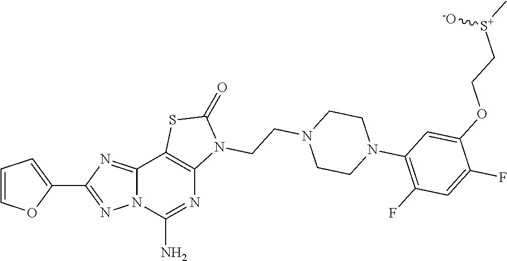 | (+)-5-amino-3-(2-(4-(2,4- difluoro-5-(2- (methylsulfinyl)ethoxy) phenyl)piperazin-1-yl)ethyl)- 8-(furan-2-yl)thiazolo[5,4- e][1,2,4]triazolo[1,5- c]pyrimidin-2(3H)-one | 604.65 |
| 8b | 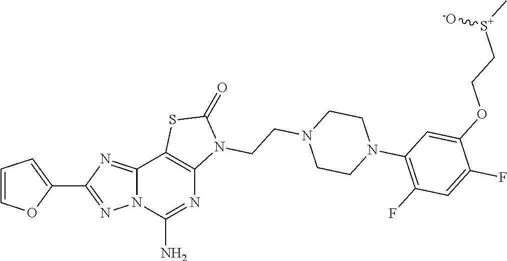 | (−)-5-amino-3-(2-(4-(2,4- difluoro-5-(2- (methylsulfinyl)ethoxy) phenyl)piperazin-1-yl)ethyl)- 8-(furan-2-yl)thiazolo[5,4- e][1,2,4]triazolo[1,5- c]pyrimidin-2(3H)-one | 604.65 |
| 9 | 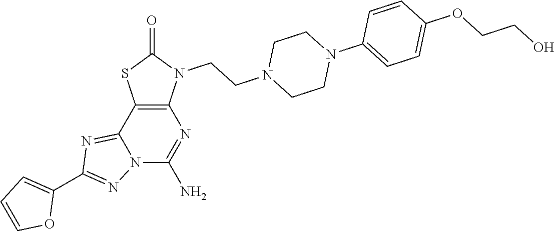 [SMILES: Nc1nc2c(sc(=O)n2CCN2CCN(c3ccc(OCCO)cc3)CC2)c2nc(-c3ccco3)nn12] | 5-amino-8-(furan-2-yl)-3- (2-(4-(4-(2-hydroxyethoxy) phenyl)piperazin-1- yl)ethyl)thiazolo[5,4- e][1,2,4]triazolo[1,5- c]pyrimidin-2(3H)-one | 522.58 |
| 10 | 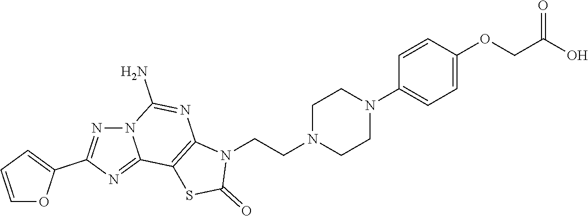 [SMILES: Nc1nc2c(sc(=O)n2CCN2CCN(c3ccc(OCC(=O)O)cc3)CC2)c2nc(-c3ccco3)nn12] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1- yl)phenoxy)acetic acid | 536.56 |
| 11 | 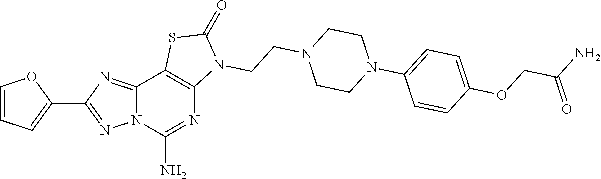 [SMILES: NC(=O)COc1ccc(N2CCN(CCn3c(=O)sc4c3nc(N)n3nc(-c5ccco5)nc43)CC2)cc1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1- yl)phenoxy)acetamide | 535.58 |
| 12 | 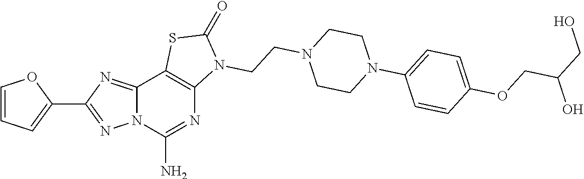 [SMILES: Nc1nc2c(sc(=O)n2CCN2CCN(c3ccc(OCC(O)CO)cc3)CC2)c2nc(-c3ccco3)nn12] | 5-amino-3-(2-(4-(4-(2,3- dihydroxypropoxy)phenyl) piperazin-1-yl)ethyl)-8- (furan-2-yl)thiazolo[5,4- e][1,2,4]triazolo[1,5- c]pyrimidin-2(3H)-one | 552.61 |
| 13 | 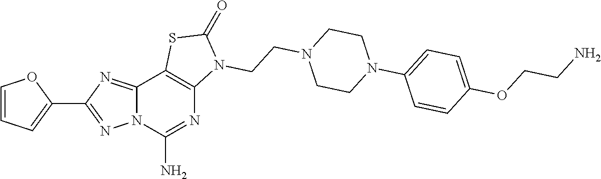 [SMILES: NCCOc1ccc(N2CCN(CCn3c(=O)sc4c3nc(N)n3nc(-c5ccco5)nc43)CC2)cc1] | 5-amino-3-(2-(4-(4-(2- aminoethoxy)phenyl) piperazin-1-yl)ethyl)-8- (furan-2-yl)thiazolo[5,4-e] [1,2,4]triazolo[1,5- c]pyrimidin-2(3H)-one | 521.60 |
| 14 | 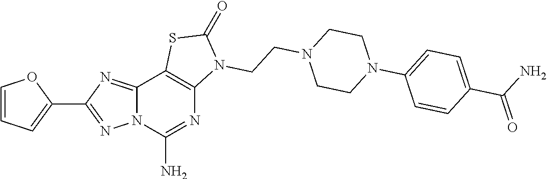 [SMILES: NC(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4-e] [1,2,4]triazolo[1,5- c]pyrimidin-3(2H)-yl) ethyl)piperazin-1- yl)benzamide | 505.55 |
| 15 | 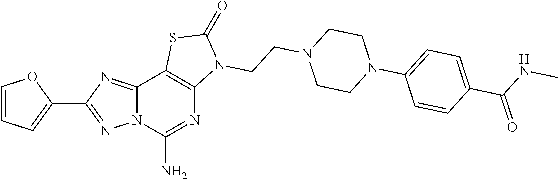 [SMILES: CNC(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- methylbenzamide | 519.58 |
| 16 | 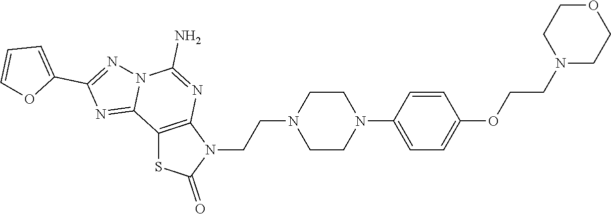 [SMILES: Nc1nc2c(sc(=O)n2CCN2CCN(c3ccc(OCCN4CCOCC4)cc3)CC2)c2nc(-c3ccco3)nn12] | 5-amino-8-(furan-2-yl)-3- (2-(4-(4-(2- morpholinoethoxy)phenyl) piperazin-1- yl)ethyl)thiazolo[5,4- e][1,2,4]triazolo[1,5- c]pyrimidin-2(3H)-one | 591.68 |
| 17 | 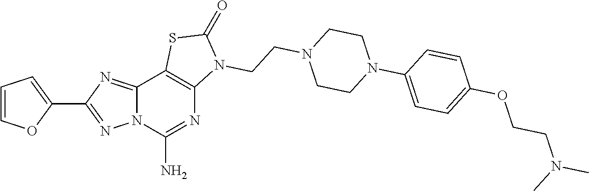 [SMILES: CN(C)CCOc1ccc(N2CCN(CCn3c(=O)sc4c3nc(N)n3nc(-c5ccco5)nc43)CC2)cc1] | 5-amino-3-(2-(4-(4-(2- (dimethylamino)ethoxy) phenyl)piperazin-1-yl)ethyl)- 8-(furan-2-yl)thiazolo[5,4- e][1,2,4]triazolo[1,5- c]pyrimidin-2(3H)-one | 549.65 |
| 18 | 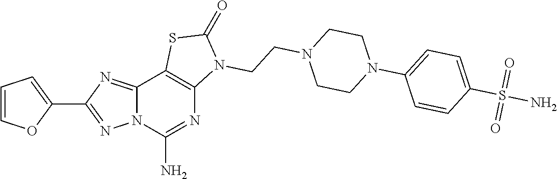 [SMILES: Nc1nc2c(sc(=O)n2CCN2CCN(c3ccc(S(N)(=O)=O)cc3)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1- yl)benzenesulfonamide | 541.61 |
| 19 | 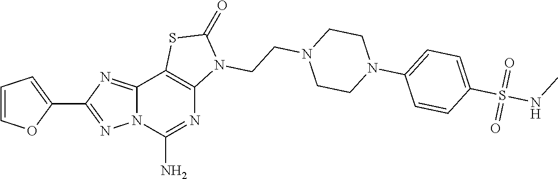 [SMILES: CNS(=O)(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4- e][1,2,4]triazolo[1,5-c] pyrimidin-3(2H)-yl)ethyl) piperazin-1-yl)-N- methylbenzenesulfonamide | 555.63 |
| 20 | 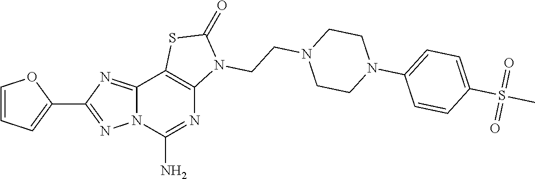 [SMILES: CS(=O)(=O)c1ccc(N2CCN(CCn3c(=O)sc4c3nc(N)n3nc(-c5ccco5)nc43)CC2)cc1] | 5-amino-8-(furan-2-yl)-3- (2-(4-(4-(methylsulfonyl) phenyl)piperazin-1- yl)ethyl)thiazolo[5,4- e][1,2,4]triazolo[1,5- c]pyrimidin-2(3H)-one | 540.62 |
| 21 | 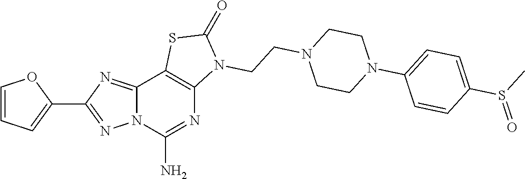 [SMILES: CS(=O)c1ccc(N2CCN(CCn3c(=O)sc4c3nc(N)n3nc(-c5ccco5)nc43)CC2)cc1] | 5-amino-8-(furan-2-yl)-3- (2-(4-(4-(methylsulfinyl) phenyl)piperazin-1- yl)ethyl)thiazolo[5,4- e][1,2,4]triazolo[1,5- c]pyrimidin-2(3H)-one | 524.62 |
| 22 | 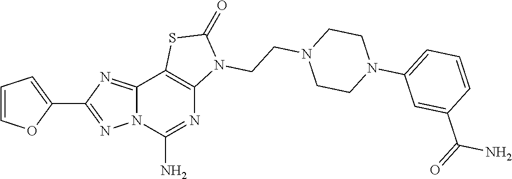 [SMILES: NC(=O)c1cccc(N2CCN(CCn3c(=O)sc4c3nc(N)n3nc(-c5ccco5)nc43)CC2)c1] | 3-(4-(2-(5-amino-8-(furan- 2-yl)-2-oxothiazolo[5,4- e][1,2,4]triazolo[1,5- c]pyrimidin-3(2H)- yl)ethyl)piperazin-1- yl)benzamide | 505.55 |
| 23 | 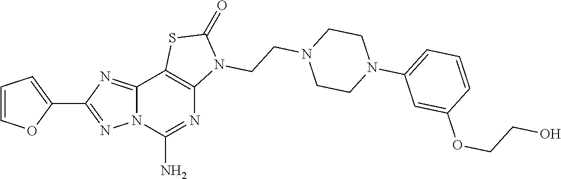 [SMILES: Nc1nc2c(sc(=O)n2CCN2CCN(c3cccc(OCCO)c3)CC2)c2nc(-c3ccco3)nn12] | 5-amino-8-(furan-2-yl)-3- (2-(4-(3-(2-hydroxyethoxy) phenyl)piperazin-1- yl)ethyl)thiazolo[5,4- e][1,2,4]triazolo[1,5- c]pyrimidin-2(3H)-one | 522.58 |
| 24 | 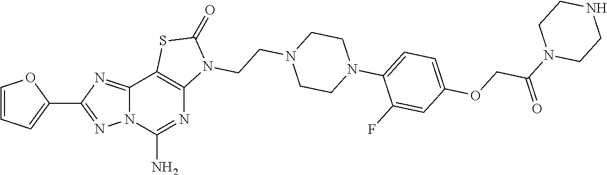 [SMILES: Nc1nc2c(sc(=O)n2CCN2CCN(c3ccc(OCC(=O)N4CCNCC4)cc3F)CC2)c2nc(-c3ccco3)nn12] | 5-amino-3-(2-(4-(2-fluoro- 4-(2-oxo-2-(piperazin-1- yl)ethoxy)phenyl)piperazin- 1-yl)ethyl)-8-(furan-2- yl)thiazolo[5,4- e][1,2,4]triazolo[1,5- c]pyrimidin-2(3H)-one | 622.67 |
| 25 | 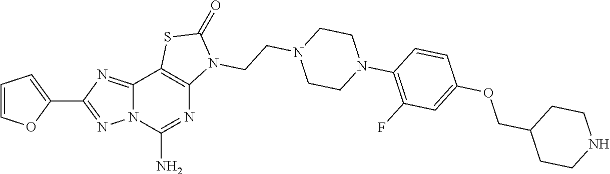 [SMILES: Nc1nc2c(sc(=O)n2CCN2CCN(c3ccc(OCC4CCNCC4)cc3F)CC2)c2nc(-c3ccco3)nn12] | 5-amino-3-(2-(4-(2-fluoro- 4-(piperidin-4-ylmethoxy) phenyl)piperazin-1- yl)ethyl)-8-(furan-2- yl)thiazolo[5,4- e][1,2,4]triazolo[1,5- c]pyrimidin-2(3H)-one | 593.68 |
| 26 | 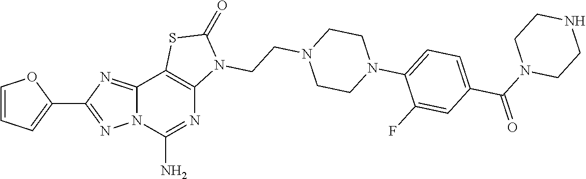 [SMILES: Nc1nc2c(sc(=O)n2CCN2CCN(c3ccc(C(=O)N4CCNCC4)cc3F)CC2)c2nc(-c3ccco3)nn12] | 5-amino-3-(2-(4-(2-fluoro- 4-(piperazine-1-carbonyl) phenyl)piperazin-1- yl)ethyl)-8-(furan-2- yl)thiazolo[5,4- e][1,2,4]triazolo[1,5- c]pyrimidin-2(3H)-one | 592.65 |
| 27 | 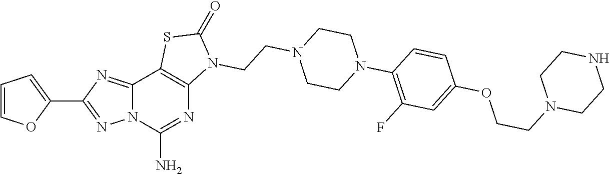 [SMILES: Nc1nc2c(sc(=O)n2CCN2CCN(c3ccc(OCCN4CCNCC4)cc3F)CC2)c2nc(-c3ccco3)nn12] | 5-amino-3-(2-(4-(2-fluoro- 4-(2-(piperazin-1- yl)ethoxy)phenyl)piperazin- 1-yl)ethyl)-8-(furan-2- yl)thiazolo[5,4- e][1,2,4]triazolo[1,5- c]pyrimidin-2(3H)-one | 608.69 |
| 28 | 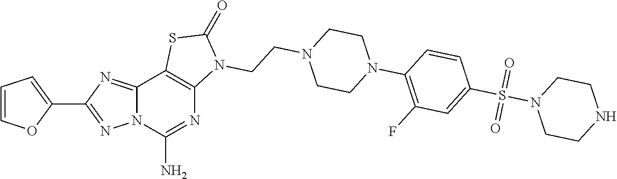 [SMILES: Nc1nc2c(sc(=O)n2CCN2CCN(c3ccc(S(=O)(=O)N4CCNCC4)cc3F)CC2)c2nc(-c3ccco3)nn12] | 5-amino-3-(2-(4-(2-fluoro- 4-(piperazin-1- ylsulfonyl)phenyl)piperazin- 1-yl)ethyl)-8-(furan-2- yl)thiazolo[5,4- e][1,2,4]triazolo[1,5- c]pyrimidin-2(3H)-one | 628.70 |
| 29 | 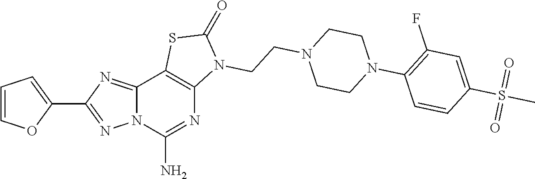 [SMILES: CS(=O)(=O)c1ccc(N2CCN(CCn3c(=O)sc4c3nc(N)n3nc(-c5ccco5)nc43)CC2)c(F)c1] | 5-amino-3-(2-(4-(2-fluoro- 4-(methylsulfonyl)phenyl) piperazin-1-yl)ethyl)-8- (furan-2-yl)thiazolo[5,4- e][1,2,4]triazolo[1,5- c]pyrimidin-2(3H)-one | 558.61 |
| 30 | 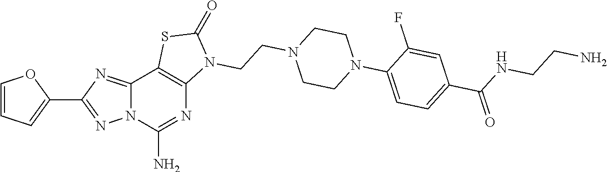 [SMILES: NCCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-aminoethyl)-3- fluorobenzamide | 566.61 |
| 31 | 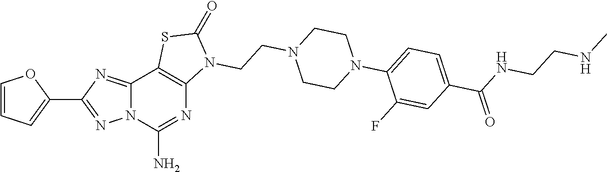 [SMILES: CNCCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- (methylamino)ethyl) benzamide | 580.64 |
| 32 | 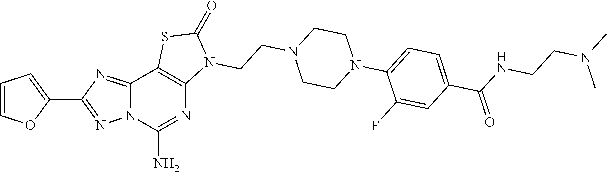 [SMILES: CN(C)CCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)-3- fluorobenzamide | 594.66 |
| 33 | 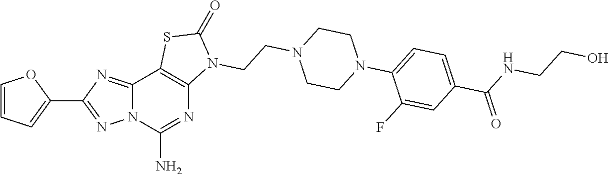 [SMILES: Nc1nc2c(sc(=O)n2CCN2CCN(c3ccc(C(=O)NCCO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- hydroxyethyl)benzamide | 567.60 |
| 34 | 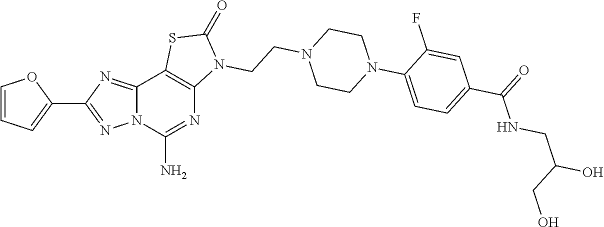 [SMILES: Nc1nc2c(sc(=O)n2CCN2CCN(c3ccc(C(=O)NCC(O)CO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2,3-dihydroxypropyl)-3- fluorobenzamide | 597.62 |
| 35 | 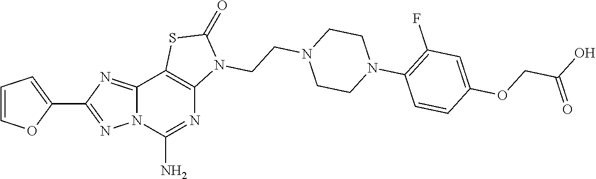 [SMILES: Nc1nc2c(sc(=O)n2CCN2CCN(c3ccc(OCC(=O)O)cc3F)CC2)c2nc(-c3ccco3)nn12] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)acetic acid | 554.55 |
| 36 | 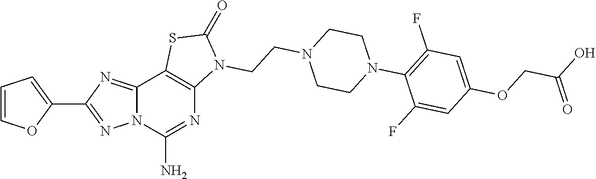 [SMILES: Nc1nc2c(sc(=O)n2CCN2CCN(c3c(F)cc(OCC(=O)O)cc3F)CC2)c2nc(-c3ccco3)nn12] | 2-(4-(4-(2-(5-amino-8- (furan-2-yl)-2-oxothiazolo [5,4-e][1,2,4]triazolo[1,5- c]pyrimidin-3(2H)-yl)ethyl) piperazin-1-yl)-3,5- difluorophenoxy)acetic acid | 572.54 |
| 37 | 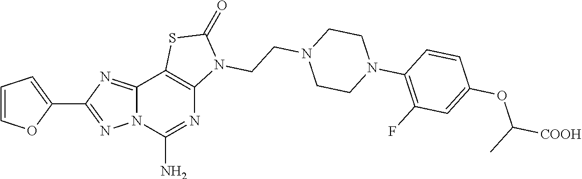 [SMILES: CC(Oc1ccc(N2CCN(CCn3c(=O)sc4c3nc(N)n3nc(-c5ccco5)nc43)CC2)c(F)c1)C(=O)O] | 2-(4-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-3- fluorophenoxy)propanoic acid | 568.58 |
| 38 | 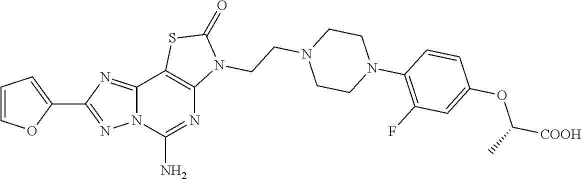 [SMILES: C[C@H](Oc1ccc(N2CCN(CCn3c(=O)sc4c3nc(N)n3nc(-c5ccco5)nc43)CC2)c(F)c1)C(=O)O] | (S)-2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)propanoic acid | 568.58 |
| 39 | 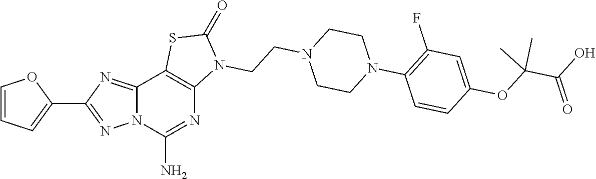 [SMILES: CC(C)(Oc1ccc(N2CCN(CCn3c(=O)sc4c3nc(N)n3nc(-c5ccco5)nc43)CC2)c(F)c1)C(=O)O] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)-2- methylpropanoic acid | 582.61 |
| 40 | 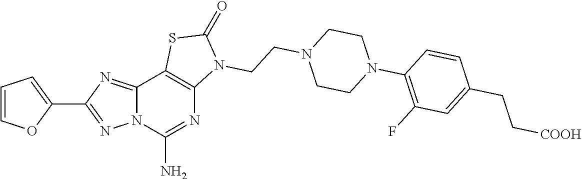 [SMILES: Nc1nc2c(sc(=O)n2CCN2CCN(c3ccc(CCC(=O)O)cc3F)CC2)c2nc(-c3ccco3)nn12] | 3-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenyl)propanoic acid | 552.58 |
| 41 | 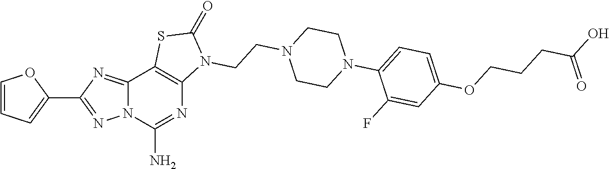 [SMILES: Nc1nc2c(sc(=O)n2CCN2CCN(c3ccc(OCCCC(=O)O)cc3F)CC2)c2nc(-c3ccco3)nn12] | 4-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)butanoic acid | 582.61 |
| 42 | 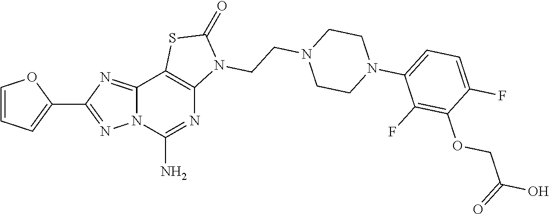 [SMILES: Nc1nc2c(sc(=O)n2CCN2CCN(c3ccc(F)c(OCC(=O)O)c3F)CC2)c2nc(-c3ccco3)nn12] | 2-(3-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,6- difluorophenoxy)acetic acid | 572.54 |
| 43 | 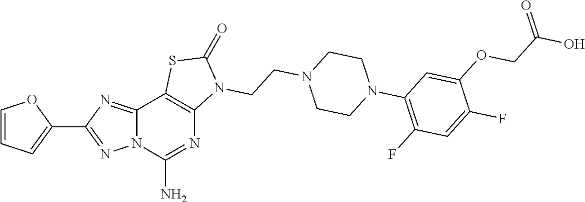 [SMILES: Nc1nc2c(sc(=O)n2CCN2CCN(c3cc(OCC(=O)O)c(F)cc3F)CC2)c2nc(-c3ccco3)nn12] | 2-(5-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,4- difluorophenoxy)acetic acid | 572.54 |
| 44 | 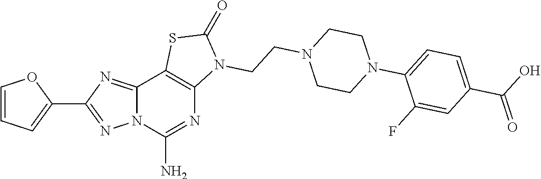 [SMILES: Nc1nc2c(sc(=O)n2CCN2CCN(c3ccc(C(=O)O)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluorobenzoic acid | 524.53 |
| 45 | 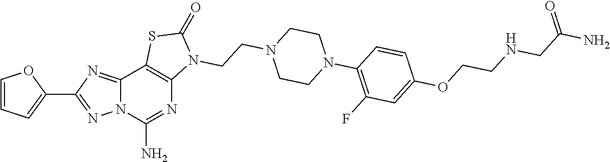 [SMILES: NC(=O)CNCCOc1ccc(N2CCN(CCn3c(=O)sc4c3nc(N)n3nc(-c5ccco5)nc43)CC2)c(F)c1] | 2-((2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)ethyl) amino)acetamide | 596.64 |
| 46 | 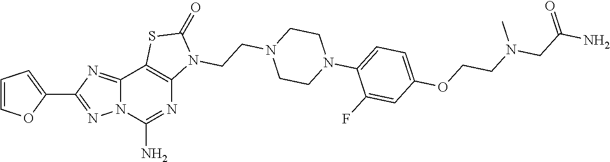 [SMILES: CN(CCOc1ccc(N2CCN(CCn3c(=O)sc4c3nc(N)n3nc(-c5ccco5)nc43)CC2)c(F)c1)CC(N)=O] | 2-((2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)ethyl)(methyl) amino)acetamide | 610.66 |
| 47 | 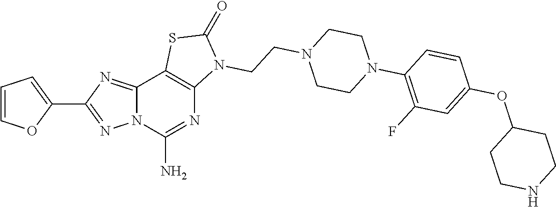 [SMILES: Nc1nc2c(sc(=O)n2CCN2CCN(c3ccc(OC4CCNCC4)cc3F)CC2)c2nc(-c3ccco3)nn12] | 5-amino-3-(2-(4-(2-fluoro- 4-(piperidin-4-yloxy) phenyl)piperazin-1-yl) ethyl)-8-(furan-2-yl) thiazolo[5,4-e][1,2,4] triazolo[1,5-c]pyrimidin- 2(3H)-one | 579.65 |
| 48 | 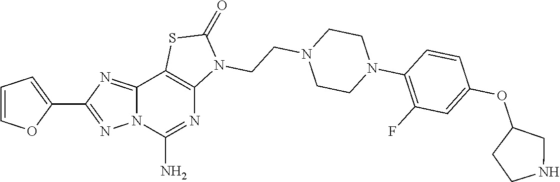 [SMILES: Nc1nc2c(sc(=O)n2CCN2CCN(c3ccc(OC4CCNC4)cc3F)CC2)c2nc(-c3ccco3)nn12] | 5-amino-3-(2-(4-(2-fluoro- 4-(pyrrolidin-3- yloxy)phenyl)piperazin-1- yl)ethyl)-8-(furan-2- yl)thiazolo[5,4- e][1,2,4]triazolo[1,5- c]pyrimidin-2(3H)-one | 565.62 |
| 49 | 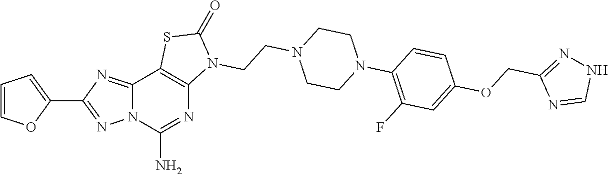 [SMILES: Nc1nc2c(sc(=O)n2CCN2CCN(c3ccc(OCc4nc[nH]n4)cc3F)CC2)c2nc(-c3ccco3)nn12] | 3-(2-(4-(4-((1H-1,2,4- triazol-3-yl)methoxy)-2- fluorophenyl)piperazin-1- yl)ethyl)-5-amino-8-(furan- 2-yl)thiazolo[5,4- e][1,2,4]triazolo[1,5- c]pyrimidin-2(3H)-one | 577.59 |
| 50 | 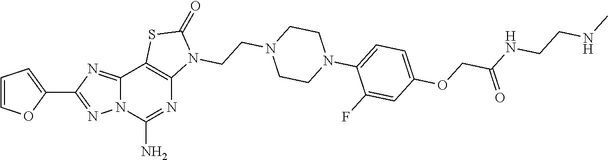 [SMILES: CNCCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)-N-(2- (methylamino)ethyl) acetamide | 610.66 |
| 51 | 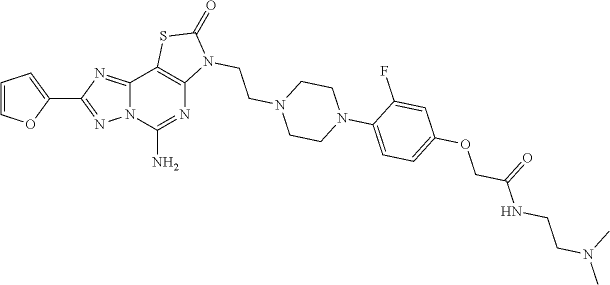 [SMILES: CN(C)CCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)-N-(2- (dimethylamino)ethyl) acetamide | 624.69 |
| 52 | 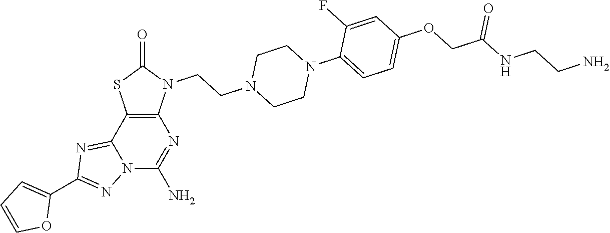 [SMILES: NCCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)-N-(2- aminoethyl)acetamide | 596.64 |
| 53 | 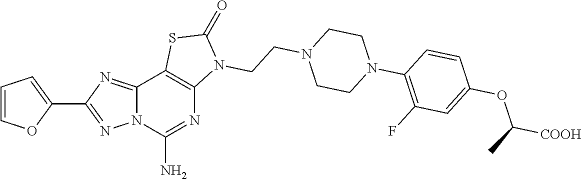 [SMILES: C[C@@H](Oc1ccc(N2CCN(CCn3c(=O)sc4c3nc(N)n3nc(-c5ccco5)nc43)CC2)c(F)c1)C(=O)O] | (R)-2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)propanoic acid | 568.58 |
| 54 | 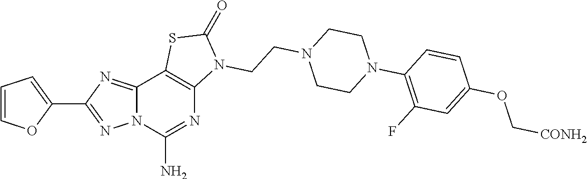 [SMILES: NC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)acetamide | 553.57 |
| 55 | 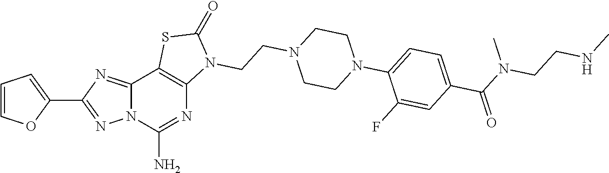 [SMILES: CNCCN(C)C(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-methyl-N-(2- (methylamino)ethyl) benzamide | 594.66 |
| 56 | 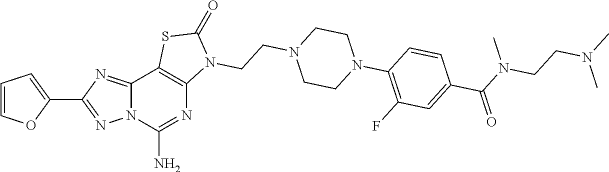 [SMILES: CN(C)CCN(C)C(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)-3- fluoro-N-methylbenzamide | 608.69 |
| 57 | 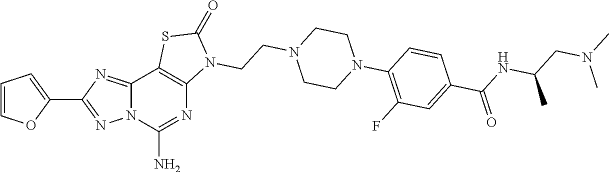 [SMILES: C[C@H](CN(C)C)NC(=O)c1ccc(N2CCN(CCn3c(=O)sc4c3nc(N)n3nc(-c5ccco5)nc43)CC2)c(F)c1] | (R)-4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-N-(1- (dimethylamino)propan-2- yl)-3-fluorobenzamide | 608.69 |
| 58 | 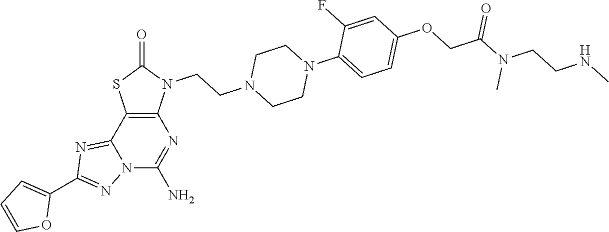 [SMILES: CNCCN(C)C(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)-N-methyl- N-(2-(methylamino)ethyl) acetamide | 624.69 |
| 59 | 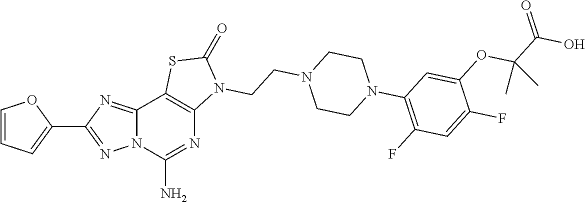 [SMILES: CC(C)(Oc1cc(N2CCN(CCn3c(=O)sc4c3nc(N)n3nc(-c5ccco5)nc43)CC2)c(F)cc1F)C(=O)O] | 2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)-2- methylpropanoic acid | 600.60 |
| 60 | 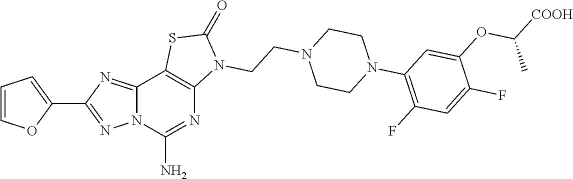 [SMILES: C[C@H](Oc1cc(N2CCN(CCn3c(=O)sc4c3nc(N)n3nc(-c5ccco5)nc43)CC2)c(F)cc1F)C(=O)O] | (S)-2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)propanoic acid | 586.57 |
| 61 | 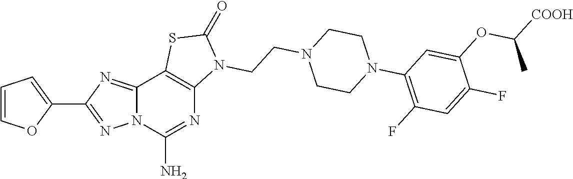 [SMILES: C[C@@H](Oc1cc(N2CCN(CCn3c(=O)sc4c3nc(N)n3nc(-c5ccco5)nc43)CC2)c(F)cc1F)C(=O)O] | (R)-2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)propanoic acid | 586.57 |
| 62 | 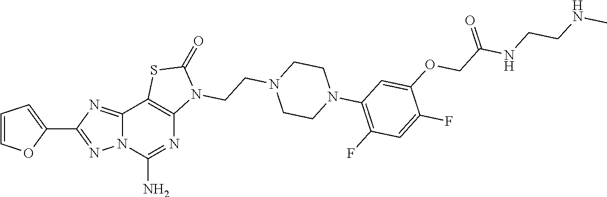 [SMILES: CNCCNC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,4- difluorophenoxy)-N-(2- (methylamino)ethyl) acetamide | 628.65 |
| 63 | 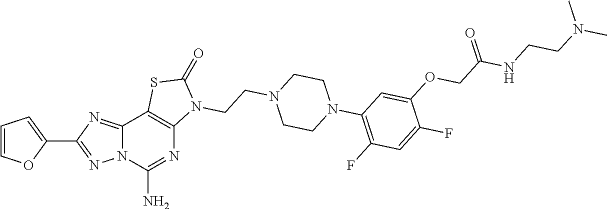 [SMILES: CN(C)CCNC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)-N-(2- (dimethylamino)ethyl) acetamide | 642.68 |
| 64 | 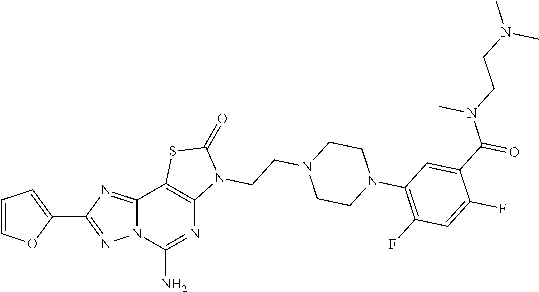 [SMILES: CN(C)CCN(C)C(=O)c1cc(N2CCN(CCn3c(=O)sc4c3nc(N)n3nc(-c5ccco5)nc43)CC2)c(F)cc1F] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)- 2,4-difluoro-N- methylbenzamide | 626.69 |
| 65 | 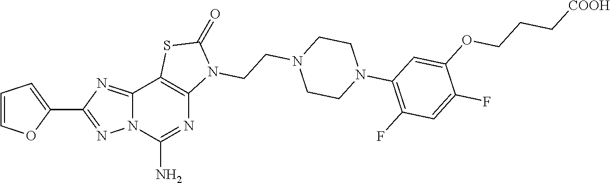 [SMILES: Nc1nc2c(sc(=O)n2CCN2CCN(c3cc(OCCCC(=O)O)c(F)cc3F)CC2)c2nc(-c3ccco3)nn12] | 4-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)butanoic acid | 600.60 |
| 66 | 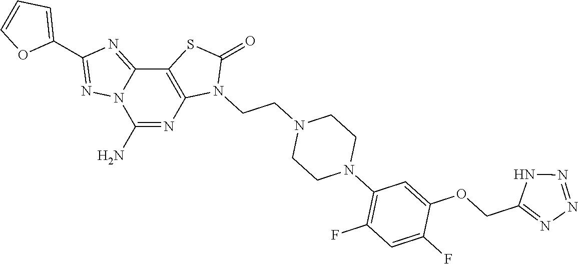 [SMILES: Nc1nc2c(sc(=O)n2CCN2CCN(c3cc(OCc4nnn[nH]4)c(F)cc3F)CC2)c2nc(-c3ccco3)nn12] | 3-(2-(4-(5-((1H-tetrazol-5- yl)methoxy)-2,4- difluorophenyl)piperazin-1- yl)ethyl)-5-amino-8-(furan- 2-yl)thiazolo[5,4- e][1,2,4]triazolo[1,5- c]pyrimidin-2(3H)-one | 596.58 |
| 67 | 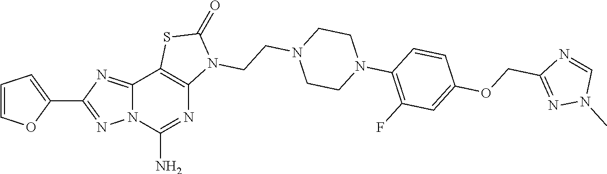 [SMILES: Cn1cnc(COc2ccc(N3CCN(CCn4c(=O)sc5c4nc(N)n4nc(-c6ccco6)nc54)CC3)c(F)c2)n1] | 5-amino-3-(2-(4-(2-fluoro- 4-((1-methyl-1H-1,2,4- triazol-3-yl)methoxy) phenyl)piperazin-1- yl)ethyl)-8-(furan-2- yl)thiazolo[5,4- e][1,2,4]triazolo[1,5- c]pyrimidin-2(3H)-one | 591.62 |
| 68 | 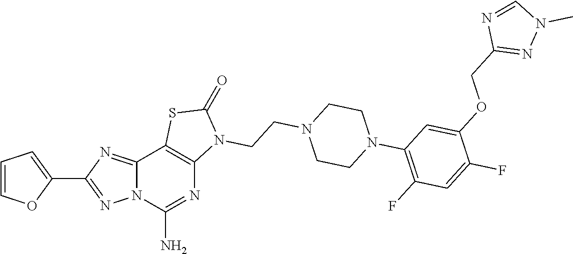 [SMILES: Cn1cnc(COc2cc(N3CCN(CCn4c(=O)sc5c4nc(N)n4nc(-c6ccco6)nc54)CC3)c(F)cc2F)n1] | 5-amino-3-(2-(4-(2,4- difluoro-5-((1-methyl-1H- 1,2,4-triazol-3-yl) methoxy)phenyl)piperazin- 1-yl)ethyl)-8-(furan-2- yl)thiazolo[5,4- e][1,2,4]triazolo[1,5- c]pyrimidin-2(3H)-one | 609.62 |
| 69 | 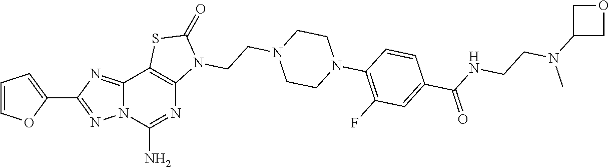 [SMILES: CN(CCNC(=O)c1ccc(N2CCN(CCn3c(=O)sc4c3nc(N)n3nc(-c5ccco5)nc43)CC2)c(F)c1)C1COC1] | 4-(4-(2-(5-amino-8-(furan- 2-yl)-2-oxothiazolo[5,4- e][1,2,4]triazolo[1,5- c]pyrimidin-3(2H)-yl) ethyl)piperazin-1-yl)-3- fluoro-N-(2-(methyl (oxetan-3-yl)amino)ethyl) benzamide | 636.70 |
| 70 | 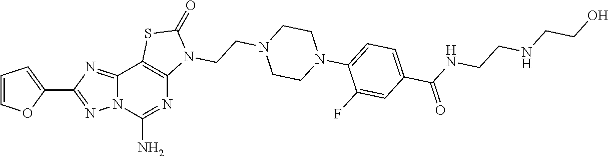 [SMILES: Nc1nc2c(sc(=O)n2CCN2CCN(c3ccc(C(=O)NCCNCCO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2-((2- hydroxyethyl)amino)ethyl) benzamide | 610.67 |
| 71 | 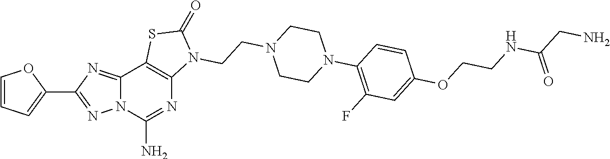 [SMILES: NCC(=O)NCCOc1ccc(N2CCN(CCn3c(=O)sc4c3nc(N)n3nc(-c5ccco5)nc43)CC2)c(F)c1] | 2-amino-N-(2-(4-(4-(2-(5- amino-8-(furan-2-yl)-2- oxothiazolo[5,4-e] [1,2,4]triazolo[1,5-c] pyrimidin-3(2H)-yl) ethyl)piperazin-1-yl)-3- fluorophenoxy)ethyl) acetamide | 596.64 |
| 72 | 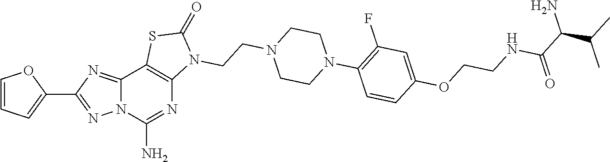 [SMILES: CC(C)[C@H](N)C(=O)NCCOc1ccc(N2CCN(CCn3c(=O)sc4c3nc(N)n3nc(-c5ccco5)nc43)CC2)c(F)c1] | (S)-2-amino-N-(2-(4-(4-(2- (5-amino-8-(furan-2-yl)-2- oxothiazolo[5,4- e][1,2,4]triazolo[1,5- c]pyrimidin-3(2H)- yl)ethyl)piperazin-1-yl)-3- fluorophenoxy)ethyl)-3- methylbutanamide | 638.72 |
| 73 | 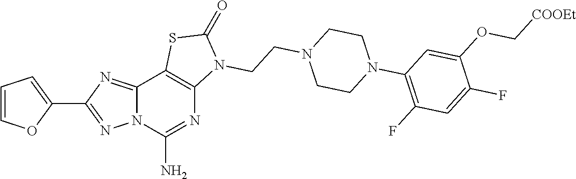 [SMILES: CCOC(=O)COc1cc(N2CCN(CCn3c(=O)sc4c3nc(N)n3nc(-c5ccco5)nc43)CC2)c(F)cc1F] | ethyl 2-(5-(4-(2-(5-amino-8- (furan-2-yl)-2- oxothiazolo[5,4-e] [1,2,4]triazolo[1,5-c] pyrimidin-3(2H)-yl) ethyl)piperazin-1-yl)-2,4- difluorophenoxy)acetate | 600.60 |
| 74 | 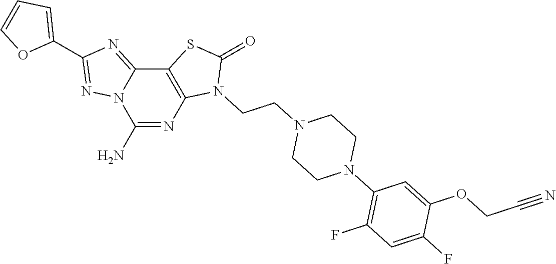 [SMILES: N#CCOc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluorophenoxy) acetonitrile | 553.54 |
| 75 | 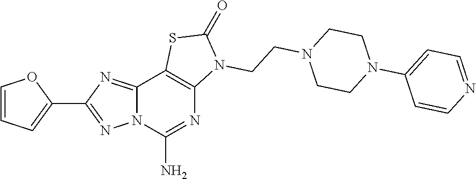 [SMILES: Nc1nc2c(sc(=O)n2CCN2CCN(c3ccncc3)CC2)c2nc(-c3ccco3)nn12] | 5-amino-8-(furan-2-yl)-3- (2-(4-(pyridin-4-yl) piperazin-1- yl)ethyl)thiazolo[5,4- e][1,2,4]triazolo[1,5- c]pyrimidin-2(3H)-one | 463.52 |
| 76 | 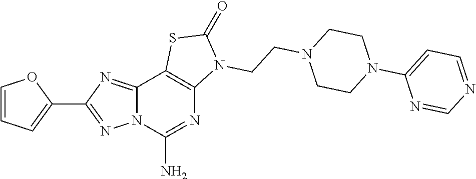 [SMILES: Nc1nc2c(sc(=O)n2CCN2CCN(c3ccncn3)CC2)c2nc(-c3ccco3)nn12] | 5-amino-8-(furan-2-yl)-3- (2-(4-(pyrimidin-4- yl)piperazin-1- yl)ethyl)thiazolo[5,4- e][1,2,4]triazolo[1,5- c]pyrimidin-2(3H)-one | 464.50 |
| 77 | 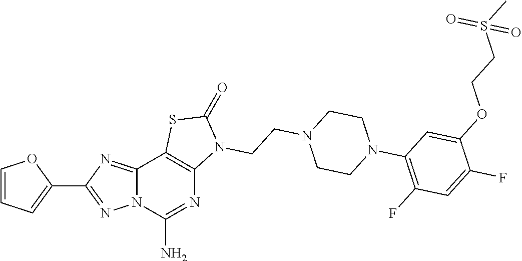 [SMILES: CS(=O)(=O)CCOc1cc(N2CCN(CCn3c(=O)sc4c3nc(N)n3nc(-c5ccco5)nc43)CC2)c(F)cc1F] | 5-amino-3-(2-(4-(2,4- difluoro-5-(2- (methylsulfonyl)ethoxy) phenyl)piperazin-1-yl)ethyl)- 8-(furan-2-yl)thiazolo[5,4- e][1,2,4]triazolo[1,5- c]pyrimidin-2(3H)-one | 620.65 |
| 78 | 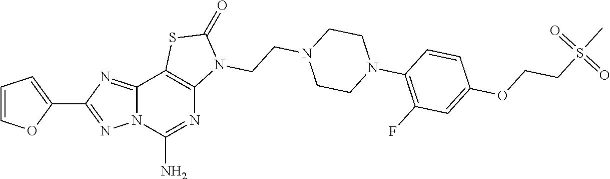 [SMILES: CS(=O)(=O)CCOc1ccc(N2CCN(CCn3c(=O)sc4c3nc(N)n3nc(-c5ccco5)nc43)CC2)c(F)c1] | 5-amino-3-(2-(4-(2-fluoro- 4-(2- (methylsulfonyl)ethoxy) phenyl)piperazin-1-yl)ethyl)- 8-(furan-2-yl)thiazolo[5,4- e][1,2,4]triazolo[1,5- c]pyrimidin-2(3H)-one | 602.66 |
| 79 | 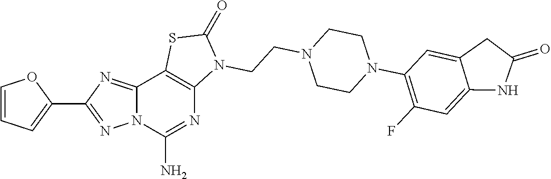 [SMILES: Nc1nc2c(sc(=O)n2CCN2CCN(c3cc4c(cc3F)NC(=O)C4)CC2)c2nc(-c3ccco3)nn12] | 5-amino-3-(2-(4-(6-fluoro- 2-oxoindolin-5- yl)piperazin-1-yl)ethyl)-8- (furan-2-yl)thiazolo[5,4- e][1,2,4]triazolo[1,5- c]pyrimidin-2(3H)-one | 535.55 |
| 80 | 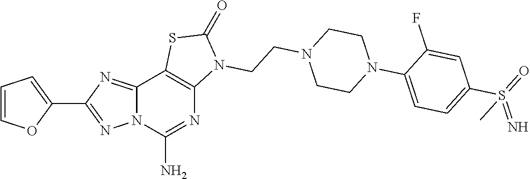 [SMILES: CS(=N)(=O)c1ccc(N2CCN(CCn3c(=O)sc4c3nc(N)n3nc(-c5ccco5)nc43)CC2)c(F)c1] | 5-amino-3-(2-(4-(2-fluoro- 4-(S- methylsulfonimidoyl)phenyl) piperazin-1-yl)ethyl)-8- (furan-2-yl)thiazolo[5,4- e][1,2,4]triazolo[1,5- c]pyrimidin-2(3H)-one | 557.62 |
| 81 | 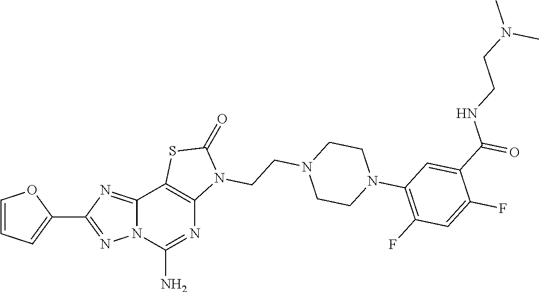 [SMILES: CN(C)CCNC(=O)c1cc(N2CCN(CCn3c(=O)sc4c3nc(N)n3nc(-c5ccco5)nc43)CC2)c(F)cc1F] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)- 2,4-difluorobenzamide | 612.65 |
| 82 | 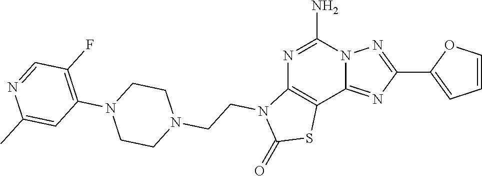 [SMILES: Cc1cc(N2CCN(CCn3c(=O)sc4c3nc(N)n3nc(-c5ccco5)nc43)CC2)c(F)cn1] | 5-amino-3-(2-(4-(5-fluoro- 2-methylpyridin-4- yl)piperazin-1-yl)ethyl)-8- (furan-2-yl)thiazolo[5,4- e][1,2,4]triazolo[1,5- c]pyrimidin-2(3H)-one | 495.53 |
| 83 | 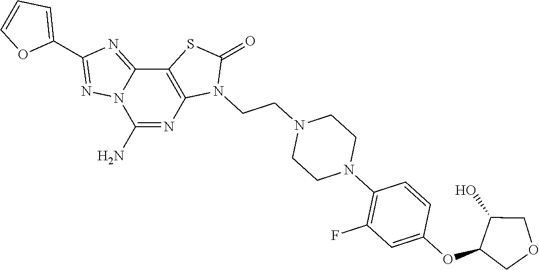 [SMILES: Nc1nc2c(sc(=O)n2CCN2CCN(c3ccc(O[C@@H]4COC[C@H]4O)cc3F)CC2)c2nc(-c3ccco3)nn12] | 5-amino-3-(2-(4-(2-fluoro- 4-(((3R,4R)-4- hydroxytetrahydrofuran-3- yl)oxy)phenyl)piperazin-1- yl)ethyl)-8-(furan-2- yl)thiazolo[5,4- e][1,2,4]triazolo[1,5- c]pyrimidin-2(3H)-one | 582.61 |
| 84 | 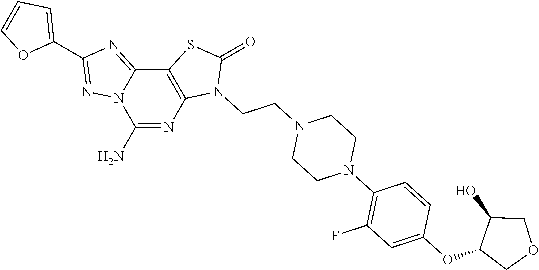 [SMILES: Nc1nc2c(sc(=O)n2CCN2CCN(c3ccc(O[C@H]4COC[C@@H]4O)cc3F)CC2)c2nc(-c3ccco3)nn12] | 5-amino-3-(2-(4-(2-fluoro- 4-(((3S,4S)-4- hydroxytetrahydrofuran-3- yl)oxy)phenyl)piperazin-1- yl)ethyl)-8-(furan-2- yl)thiazolo[5,4- e][1,2,4]triazolo[1,5- c]pyrimidin-2(3H)-one | 582.61 |
| 85 | 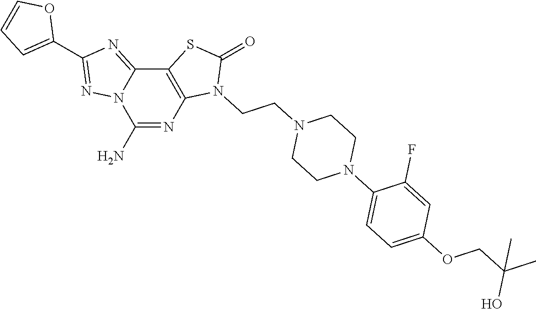 [SMILES: CC(C)(O)COc1ccc(N2CCN(CCn3c(=O)sc4c3nc(N)n3nc(-c5ccco5)nc43)CC2)c(F)c1] | 5-amino-3-(2-(4-(2-fluoro- 4-(2-hydroxy-2- methylpropoxy)phenyl) piperazin-1-yl)ethyl)-8-(furan- 2-yl)thiazolo[5,4- e][1,2,4]triazolo[1,5- c]pyrimidin-2(3H)-one | 568.62 |
| 86 | 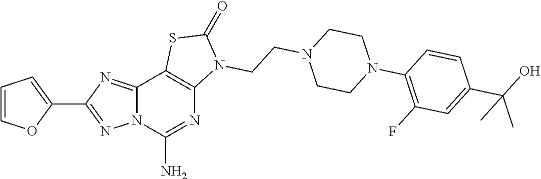 [SMILES: CC(C)(O)c1ccc(N2CCN(CCn3c(=O)sc4c3nc(N)n3nc(-c5ccco5)nc43)CC2)c(F)c1] | 5-amino-3-(2-(4-(2-fluoro- 4-(2-hydroxypropan-2- yl)phenyl)piperazin-1- yl)ethyl)-8-(furan-2- yl)thiazolo[5,4- e][1,2,4]triazolo[1,5- c]pyrimidin-2(3H)-one | 538.60 |
| 87 | 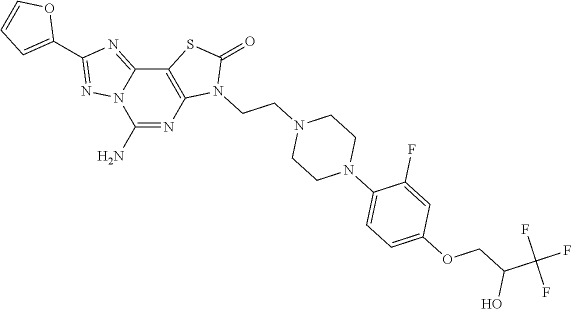 [SMILES: Nc1nc2c(sc(=O)n2CCN2CCN(c3ccc(OCC(O)C(F)(F)F)cc3F)CC2)c2nc(-c3ccco3)nn12] | 5-amino-3-(2-(4-(2-fluoro- 4-(3,3,3-trifluoro-2- hydroxypropoxy)phenyl) piperazin-1-yl)ethyl)-8-(furan- 2-yl)thiazolo[5,4- e][1,2,4]triazolo[1,5- c]pyrimidin-2(3H)-one | 608.57 |
| 88 | 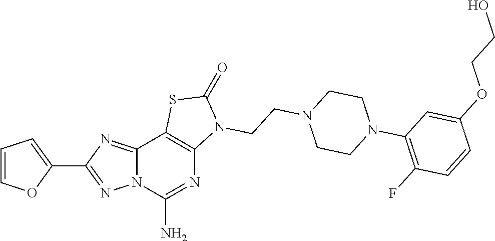 [SMILES: Nc1nc2c(sc(=O)n2CCN2CCN(c3cc(OCCO)ccc3F)CC2)c2nc(-c3ccco3)nn12] | 5-amino-3-(2-(4-(2-fluoro- 5-(2- hydroxyethoxy)phenyl) piperazin-1-yl)ethyl)-8-(furan- 2-yl)thiazolo[5,4- e][1,2,4]triazolo[1,5- c]pyrimidin-2(3H)-one | 540.57 |
| 89 | 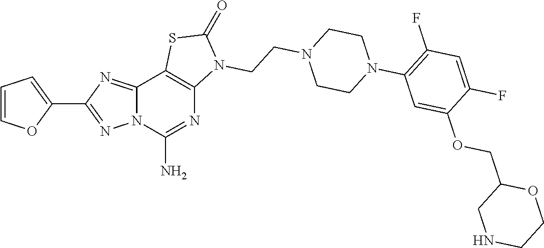 [SMILES: Nc1nc2c(sc(=O)n2CCN2CCN(c3cc(OCC4CNCCO4)c(F)cc3F)CC2)c2nc(-c3ccco3)nn12] | 5-amino-3-(2-(4-(2,4- difluoro-5-(morpholin-2- ylmethoxy)phenyl)piperazin- 1-yl)ethyl)-8-(furan-2- yl)thiazolo[5,4- e][1,2,4]triazolo[1,5- c]pyrimidin-2(3H)-one | 613.64 |
| 90 | 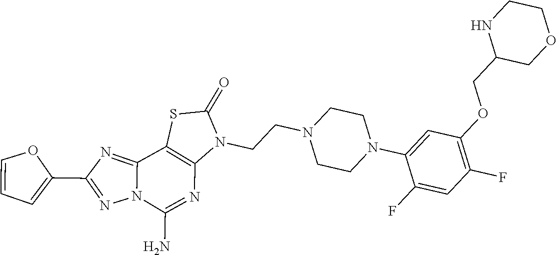 [SMILES: Nc1nc2c(sc(=O)n2CCN2CCN(c3cc(OCC4COCCN4)c(F)cc3F)CC2)c2nc(-c3ccco3)nn12] | 5-amino-3-(2-(4-(2,4- difluoro-5-(morpholin-3- ylmethoxy)phenyl)piperazin- 1-yl)ethyl)-8-(furan-2- yl)thiazolo[5,4- e][1,2,4]triazolo[1,5- c]pyrimidin-2(3H)-one | 613.64 |
| 91 | 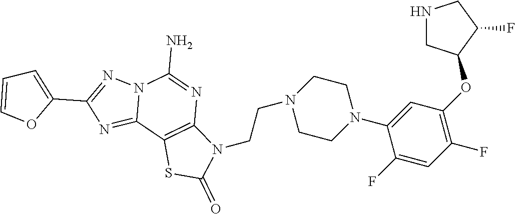 [SMILES: Nc1nc2c(sc(=O)n2CCN2CCN(c3cc(O[C@H]4CNC[C@@H]4F)c(F)cc3F)CC2)c2nc(-c3ccco3)nn12] | 5-amino-3-(2-(4-(2,4- difluoro-5-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 92 | 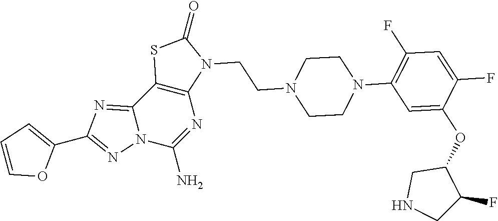 [SMILES: Nc1nc2c(sc(=O)n2CCN2CCN(c3cc(O[C@H]4CNC[C@@H]4F)c(F)cc3F)CC2)c2nc(-c3ccco3)nn12] | 5-amino-3-(2-(4-(2,4- difluoro-5-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 93 | 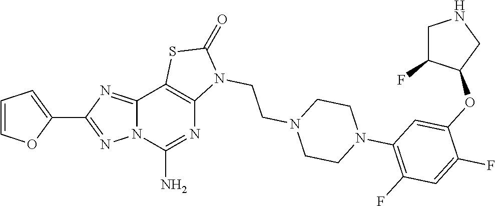 [SMILES: Nc1nc2c(sc(=O)n2CCN2CCN(c3cc(O[C@@H]4CNC[C@@H]4F)c(F)cc3F)CC2)c2nc(-c3ccco3)nn12] | 5-amino-3-(2-(4-(2,4- difluoro-5-(((3R,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 94 | 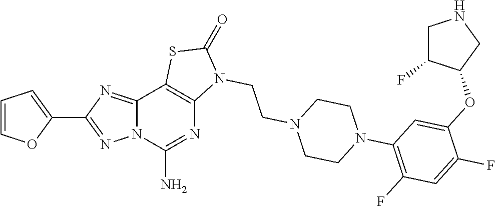 [SMILES: Nc1nc2c(sc(=O)n2CCN2CCN(c3cc(O[C@H]4CNC[C@H]4F)c(F)cc3F)CC2)c2nc(-c3ccco3)nn12] | 5-amino-3-(2-(4-(2,4- difluoro-5-(((3S,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 95 | 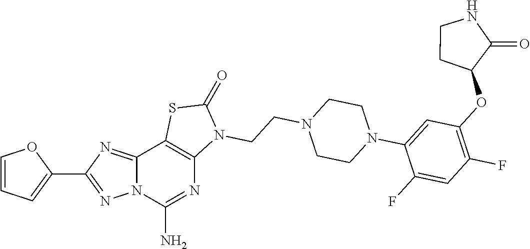 [SMILES: Nc1nc2c(sc(=O)n2CCN2CCN(c3cc(O[C@H]4CCNC4=O)c(F)cc3F)CC2)c2nc(-c3ccco3)nn12] | (S)-5-amino-3-(2-(4-(2,4- difluoro-5-((2- oxopyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 597.60 |
| 96 | 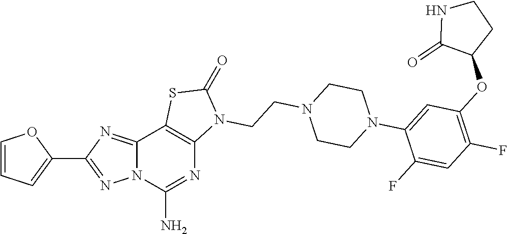 [SMILES: Nc1nc2c(sc(=O)n2CCN2CCN(c3cc(O[C@@H]4CCNC4=O)c(F)cc3F)CC2)c2nc(-c3ccco3)nn12] | (R)-5-amino-3-(2-(4-(2,4- difluoro-5-((2- oxopyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 597.60 |
| 97 | 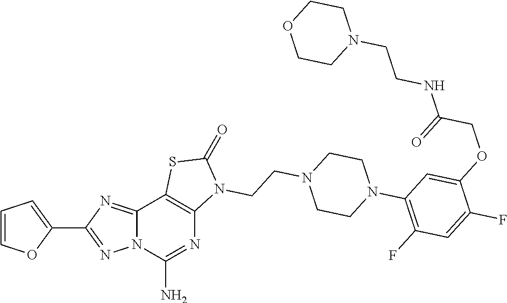 [SMILES: Nc1nc2c(sc(=O)n2CCN2CCN(c3cc(OCC(=O)NCCN4CCOCC4)c(F)cc3F)CC2)c2nc(-c3ccco3)nn12] | 2-(5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluorophenoxy)-N-(2- morpholinoethyl)acetamide | 684.72 |
| 98 | 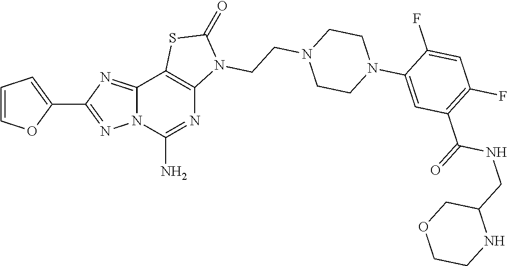 [SMILES: Nc1nc2c(sc(=O)n2CCN2CCN(c3cc(C(=O)NCC4COCCN4)c(F)cc3F)CC2)c2nc(-c3ccco3)nn12] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-2,4- difluoro-N-(morpholin-3- ylmethyl)benzamide | 640.66 |
| 99 | 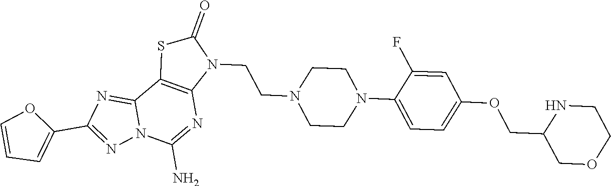 [SMILES: Nc1nc2c(sc(=O)n2CCN2CCN(c3ccc(OCC4COCCN4)cc3F)CC2)c2nc(-c3ccco3)nn12] | 5-amino-3-(2-(4-(2-fluoro- 4-(morpholin-3- ylmethoxy)phenyl)piperazin- 1-yl)ethyl)-8-(furan-2- yl)thiazolo[5,4- e][1,2,4]triazolo[1,5- c]pyrimidin-2(3H)-one | 595.65 |
| 100 | 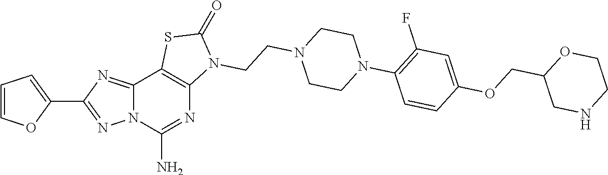 [SMILES: Nc1nc2c(sc(=O)n2CCN2CCN(c3ccc(OCC4CNCCO4)cc3F)CC2)c2nc(-c3ccco3)nn12] | 5-amino-3-(2-(4-(2-fluoro- 4-(morpholin-2- ylmethoxy)phenyl)piperazin- 1-yl)ethyl)-8-(furan-2- yl)thiazolo[5,4- e][1,2,4]triazolo[1,5- c]pyrimidin-2(3H)-one | 595.65 |
| 101 | 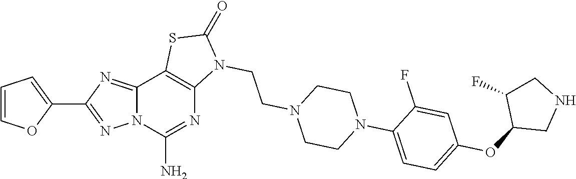 [SMILES: Nc1nc2c(sc(=O)n2CCN2CCN(c3ccc(O[C@@H]4CNC[C@H]4F)cc3F)CC2)c2nc(-c3ccco3)nn12] | 5-amino-3-(2-(4-(2-fluoro- 4-(((3R,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 102 | 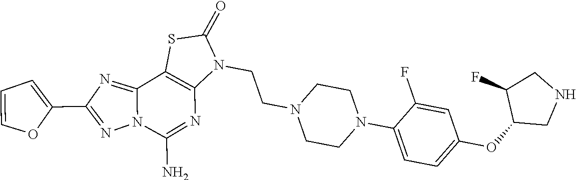 [SMILES: Nc1nc2c(sc(=O)n2CCN2CCN(c3ccc(O[C@H]4CNC[C@@H]4F)cc3F)CC2)c2nc(-c3ccco3)nn12] | 5-amino-3-(2-(4-(2-fluoro- 4-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 103 | 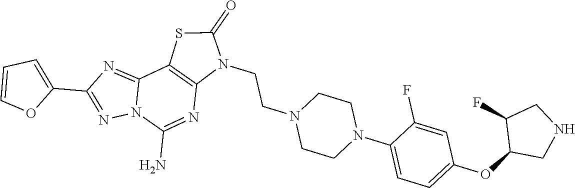 [SMILES: Nc1nc2c(sc(=O)n2CCN2CCN(c3ccc(O[C@@H]4CNC[C@@H]4F)cc3F)CC2)c2nc(-c3ccco3)nn12] | 5-amino-3-(2-(4-(2-fluoro- 4-(((3R,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 104 | 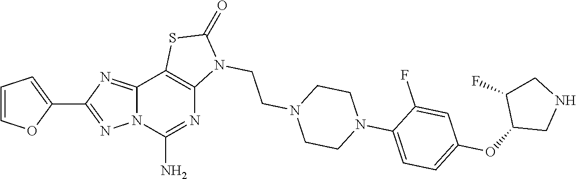 [SMILES: Nc1nc2c(sc(=O)n2CCN2CCN(c3ccc(O[C@H]4CNC[C@H]4F)cc3F)CC2)c2nc(-c3ccco3)nn12] | 5-amino-3-(2-(4-(2-fluoro- 4-(((3S,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 105 | 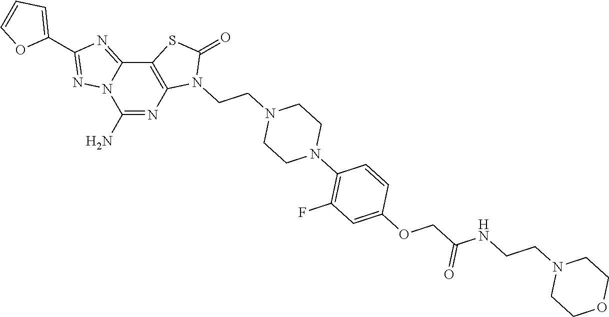 [SMILES: Nc1nc2c(sc(=O)n2CCN2CCN(c3ccc(OCC(=O)NCCN4CCOCC4)cc3F)CC2)c2nc(-c3ccco3)nn12] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)-N-(2- morpholinoethyl)acetamide | 666.73 |
| 106 | 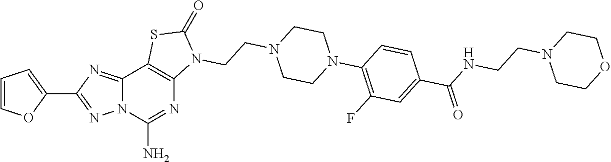 [SMILES: Nc1nc2c(sc(=O)n2CCN2CCN(c3ccc(C(=O)NCCN4CCOCC4)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- morpholinoethyl)benzamide | 636.70 |
| 107 | 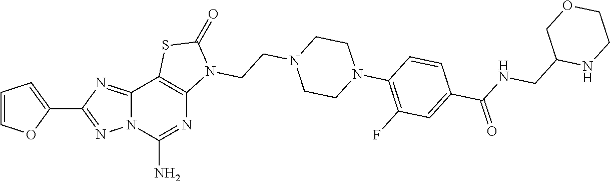 [SMILES: Nc1nc2c(sc(=O)n2CCN2CCN(c3ccc(C(=O)NCC4COCCN4)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(morpholin-3- ylmethyl)benzamide | 622.67 |
| 108 | 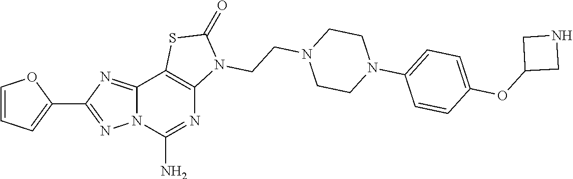 [SMILES: Nc1nc2c(sc(=O)n2CCN2CCN(c3ccc(OC4CNC4)cc3)CC2)c2nc(-c3ccco3)nn12] | 5-amino-3-(2-(4-(4- (azetidin-3- yloxy)phenyl)piperazin-1- yl)ethyl)-8-(furan-2- yl)thiazolo[5,4- e][1,2,4]triazolo[1,5- c]pyrimidin-2(3H)-one | 533.61 |
| 109 | 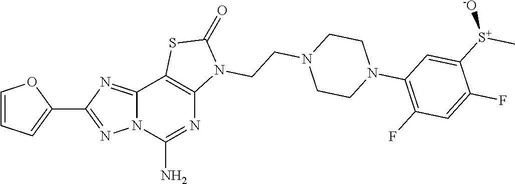 | (S)-5-amino-3-(2-(4-(2,4- difluoro-5- (methylsulfinyl)phenyl) piperazin-1-yl)ethyl)-8-(furan- 2-yl)thiazolo[5,4- e][1,2,4]triazolo[1,5- c]pyrimidin-2(3H)-one | 560.60 |
| 110 | 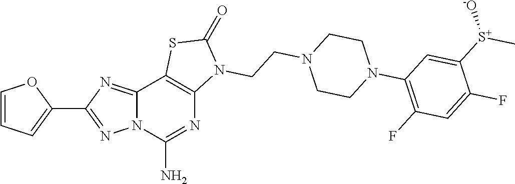 | (R)-5-amino-3-(2-(4-(2,4- difluoro-5- (methylsulfinyl)phenyl) piperazin-1-yl)ethyl)-8-(furan- 2-yl)thiazolo[5,4- e][1,2,4]triazolo[1,5- c]pyrimidin-2(3H)-one | 560.60 |
| 111 | 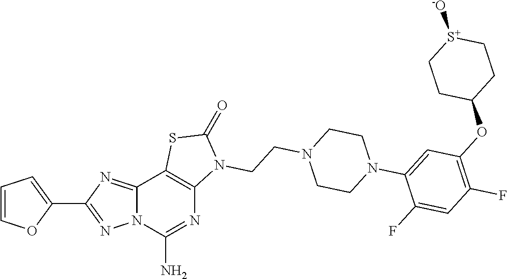 | 5-amino-3-(2-(4-(2,4- difluoro-5-(((1s,4s)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 630.69 |
| 112 | 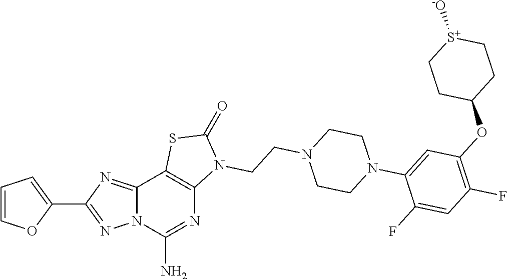 | 5-amino-3-(2-(4-(2,4- difluoro-5-(((1r,4r)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 630.69 |
| 113 | 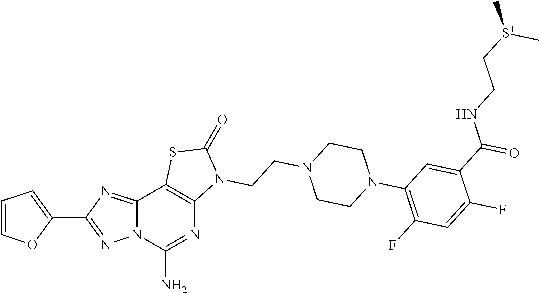 | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-(2- (methylsulfinyl)ethyl) benzamide | 631.68 |
| 114 | 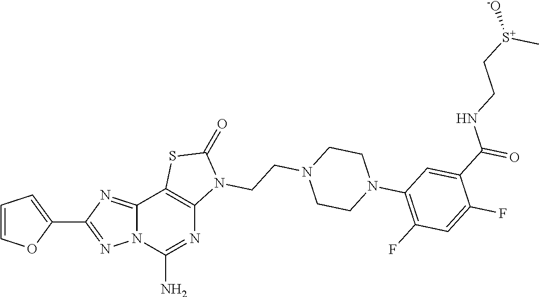 | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-(2- (methylsulfinyl)ethyl) benzamide | 631.68 |
| 115 | 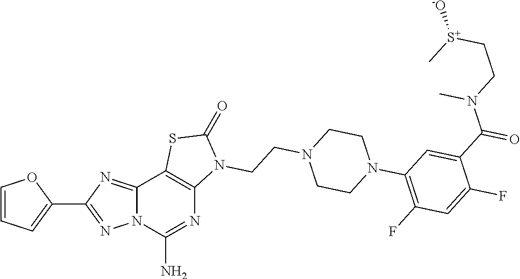 | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-methyl-N-(2- (methylsulfinyl)ethyl) benzamide | 645.70 |
| 116 | 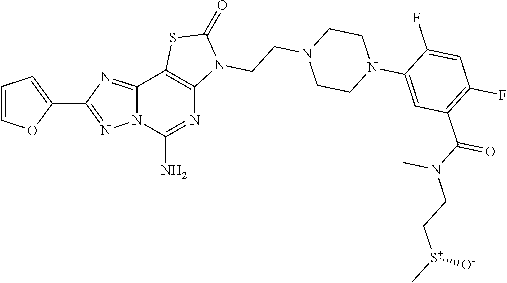 [SMILES: CN(CC[S@@+](C)[O-])C(=O)c1cc(N2CCN(CCn3c(=O)sc4c3nc(N)n3nc(-c5ccco5)nc43)CC2)c(F)cc1F] | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-methyl-N-(2- (methylsulfinyl)ethyl) benzamide | 645.70 |
| 117 | 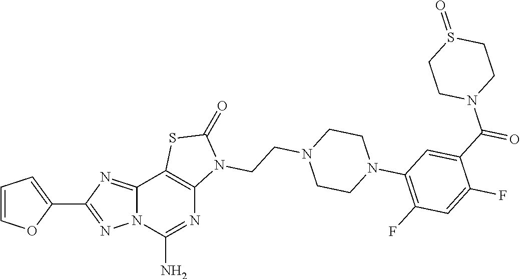 [SMILES: Nc1nc2c(sc(=O)n2CCN2CCN(c3cc(C(=O)N4CCS(=O)CC4)c(F)cc3F)CC2)c2nc(-c3ccco3)nn12] | 5-amino-3-(2-(4-(2,4- difluoro-5-(1- oxidothiomorpholine-4- carbonyl)phenyl)piperazin- 1-yl)ethyl)-8-(furan-2- yl)thiazolo[5,4- e][1,2,4]triazolo[1,5- c]pyrimidin-2(3H)-one | 643.69 |
| 118 | 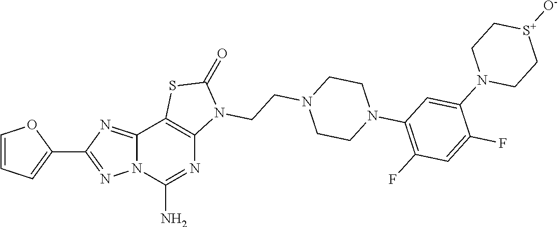 [SMILES: Nc1nc2c(sc(=O)n2CCN2CCN(c3cc(N4CC[S+]([O-])CC4)c(F)cc3F)CC2)c2nc(-c3ccco3)nn12] | 5-amino-3-(2-(4-(2,4- difluoro-5-(1- oxidothiomorpholino)phenyl) piperazin-1-yl)ethyl)-8- (furan-2-yl)thiazolo[5,4- e][1,2,4]triazolo[1,5- c]pyrimidin-2(3H)-one | 615.68 |
| 119 | 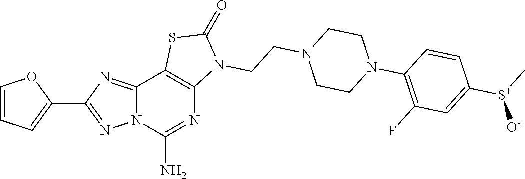 [SMILES: C[S@@+]([O-])c1ccc(N2CCN(CCn3c(=O)sc4c3nc(N)n3nc(-c5ccco5)nc43)CC2)c(F)c1] | (R)-5-amino-3-(2-(4-(2- fluoro-4- (methylsulfinyl)phenyl) piperazin-1-yl)ethyl)-8-(furan- 2-yl)thiazolo[5,4- e][1,2,4]triazolo[1,5- c]pyrimidin-2(3H)-one | 542.61 |
| 120 | 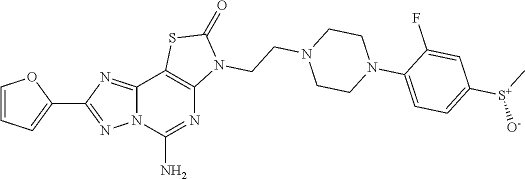 [SMILES: C[S@+]([O-])c1ccc(N2CCN(CCn3c(=O)sc4c3nc(N)n3nc(-c5ccco5)nc43)CC2)c(F)c1] | (S)-5-amino-3-(2-(4-(2- fluoro-4- (methylsulfinyl)phenyl) piperazin-1-yl)ethyl)-8-(furan- 2-yl)thiazolo[5,4- e][1,2,4]triazolo[1,5- c]pyrimidin-2(3H)-one | 542.61 |
| 121 | 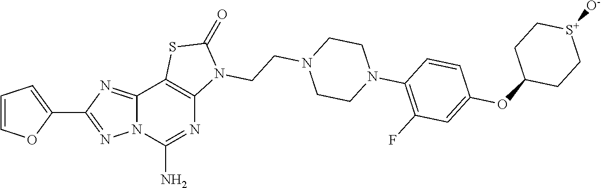 [SMILES: Nc1nc2c(sc(=O)n2CCN2CCN(c3ccc(O[C@H]4CC[S@@+]([O-])CC4)cc3F)CC2)c2nc(-c3ccco3)nn12] | 5-amino-3-(2-(4-(2-fluoro- 4-(((1s,4s)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 612.70 |
| 122 | 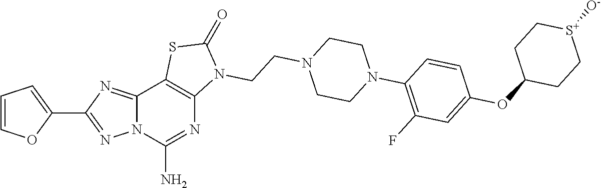 [SMILES: Nc1nc2c(sc(=O)n2CCN2CCN(c3ccc(O[C@H]4CC[S@+]([O-])CC4)cc3F)CC2)c2nc(-c3ccco3)nn12] | 5-amino-3-(2-(4-(2-fluoro- 4-(((1r,4r)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 612.70 |
| 123 | 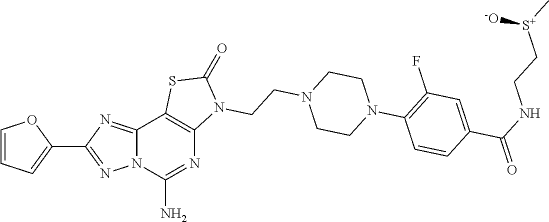 | (S)-4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluoro-N-(2- (methylsulfinyl)ethyl) benzamide | 613.69 |
| 124 | 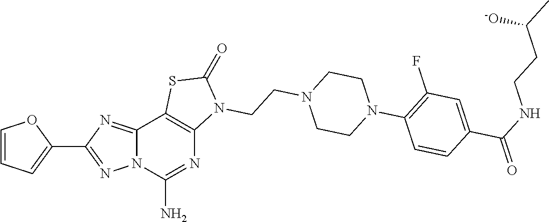 | (R)-4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluoro-N-(2- (methylsulfinyl)ethyl) benzamide | 613.69 |
| 125 | 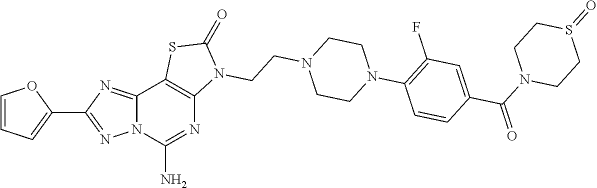 [SMILES: Nc1nc2c(sc(=O)n2CCN2CCN(c3ccc(C(=O)N4CCS(=O)CC4)cc3F)CC2)c2nc(-c3ccco3)nn12] | 5-amino-3-(2-(4-(2-fluoro- 4-(1-oxidothiomorpholine- 4- carbonyl)phenyl)piperazin- 1-yl)ethyl)-8-(furan-2- yl)thiazolo[5,4- e][1,2,4]triazolo[1,5- c]pyrimidin-2(3H)-one | 625.70 |
| 126 | 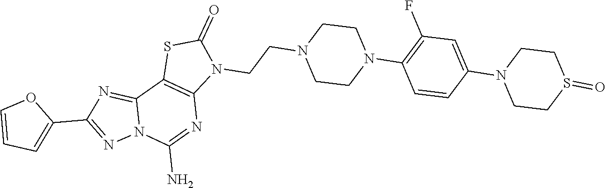 [SMILES: Nc1nc2c(sc(=O)n2CCN2CCN(c3ccc(N4CCS(=O)CC4)cc3F)CC2)c2nc(-c3ccco3)nn12] | 5-amino-3-(2-(4-(2-fluoro- 4-(1- oxidothiomorpholino)phenyl) piperazin-1-yl)ethyl)-8- (furan-2-yl)thiazolo[5,4- e][1,2,4]triazolo[1,5- c]pyrimidin-2(3H)-one | 597.69 |
| 127 | 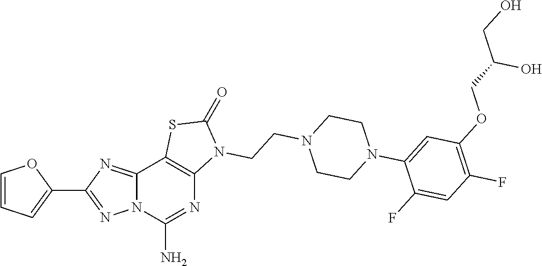 [SMILES: Nc1nc2c(sc(=O)n2CCN2CCN(c3cc(OC[C@@H](O)CO)c(F)cc3F)CC2)c2nc(-c3ccco3)nn12] | (S)-5-amino-3-(2-(4-(5-(2,3- dihydroxypropoxy)-2,4- difluorophenyl)piperazin-1- yl)ethyl)-8-(furan-2- yl)thiazolo[5,4- e][1,2,4]triazolo[1,5- c]pyrimidin-2(3H)-one | 588.59 |
| 128 | 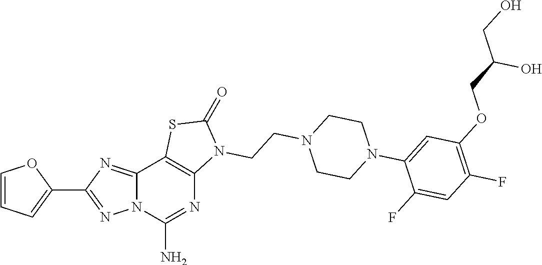 [SMILES: Nc1nc2c(sc(=O)n2CCN2CCN(c3cc(OC[C@H](O)CO)c(F)cc3F)CC2)c2nc(-c3ccco3)nn12] | (R)-5-amino-3-(2-(4-(5- (2,3-dihydroxypropoxy)- 2,4- difluorophenyl)piperazin-1- yl)ethyl)-8-(furan-2- yl)thiazolo[5,4- e][1,2,4]triazolo[1,5- c]pyrimidin-2(3H)-one | 588.59 |
| 129 | 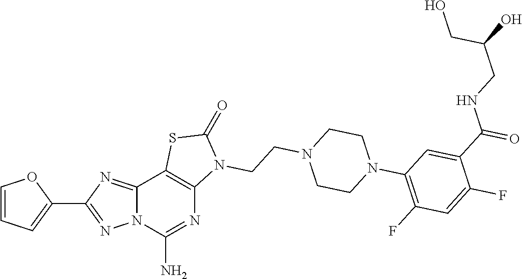 [SMILES: Nc1nc2c(sc(=O)n2CCN2CCN(c3cc(C(=O)NC[C@H](O)CO)c(F)cc3F)CC2)c2nc(-c3ccco3)nn12] | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-N- (2,3-dihydroxypropyl)-2,4- difluorobenzamide | 615.61 |
| 130 | 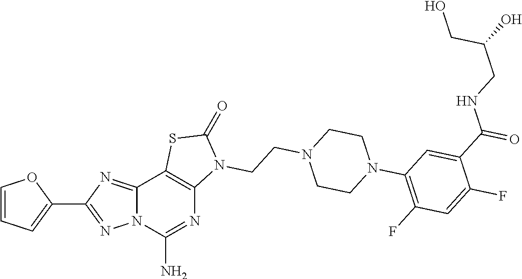 [SMILES: Nc1nc2c(sc(=O)n2CCN2CCN(c3cc(C(=O)NC[C@@H](O)CO)c(F)cc3F)CC2)c2nc(-c3ccco3)nn12] | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-N- (2,3-dihydroxypropyl)-2,4- difluorobenzamide | 615.61 |
| 131 | 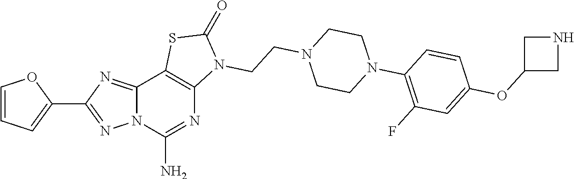 [SMILES: Nc1nc2c(sc(=O)n2CCN2CCN(c3ccc(OC4CNC4)cc3F)CC2)c2nc(-c3ccco3)nn12] | 5-amino-3-(2-(4-(4- (azetidin-3-yloxy)-2- fluorophenyl)piperazin-1- yl)ethyl)-8-(furan-2- yl)thiazolo[5,4- e][1,2,4]triazolo[1,5- c]pyrimidin-2(3H)-one | 551.60 |
| 132 | 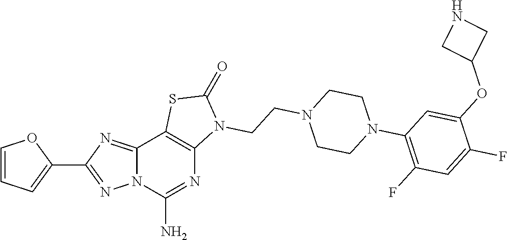 [SMILES: Nc1nc2c(sc(=O)n2CCN2CCN(c3cc(OC4CNC4)c(F)cc3F)CC2)c2nc(-c3ccco3)nn12] | 5-amino-3-(2-(4-(5- (azetidin-3-yloxy)-2,4- difluorophenyl)piperazin-1- yl)ethyl)-8-(furan-2- yl)thiazolo[5,4- e][1,2,4]triazolo[1,5- c]pyrimidin-2(3H)-one | 569.59 |
| 133 | 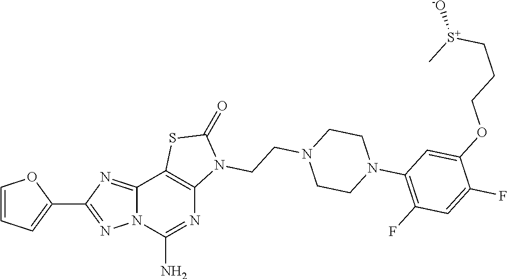 | (S)-5-amino-3-(2-(4-(2,4- difluoro-5-(3- (methylsulfinyl)propoxy) phenyl)piperazin-1-yl)ethyl)- 8-(furan-2-yl)thiazolo[5,4- e][1,2,4]triazolo[1,5- c]pyrimidin-2(3H)-one | 618.68 |

and pharmaceutically acceptable salts and solvates thereof.

In Table 1, the term “Cpd” means compound.

The compounds of Table 1 were named using ChemBioDraw® Ultra version 12.0 (PerkinElmer).

In one embodiment, the A2AR antagonist is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7);
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a) and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b).

In a specific embodiment, the A2AR antagonist is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7); and
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In preferred embodiment, the A2AR antagonist is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In another preferred embodiment, the A2AR antagonist is (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b). In one embodiment, the present invention also relates to enantiomers, salts, solvates, polymorphs, multi-component complexes and liquid crystals of compounds of Formula (I) and subformulae thereof.

In one embodiment, the present invention also relates to polymorphs and crystal habits of compounds of Formula (I) and subformulae thereof, prodrugs and isomers thereof (including optical, geometric and tautomeric isomers) and isotopically-labeled compounds of Formula (I) and subformulae thereof.

The compounds of Formula (I) and subformulae thereof may contain an asymmetric center and thus may exist as different stereoisomeric forms. Accordingly, the present invention includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

The compounds of the invention may be in the form of pharmaceutically acceptable salts. Pharmaceutically acceptable salts of the compounds of Formula (I) and subformulae thereof include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, tosylate, esylate and acetate. In a particularly preferred embodiment, the compounds of Formula (I) is under the form of a HCl salt or esylate salt.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula (I) and subformulae thereof may be prepared by one or more of these methods:
(i) by reacting the compound of Formula (I) with the desired acid;
(ii) by reacting the compound of Formula (I) with the desired base;
(iii) by removing an acid- or base-labile protecting group from a suitable precursor of the compound of Formula (I) or by ring-opening a suitable cyclic precursor, for example, a lactone or lactam, using the desired acid; or
(iv) by converting one salt of the compound of Formula (I) to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

These salts may be prepared by standard procedures, e.g. by reacting a free acid with a suitable organic or inorganic base. Where a basic group is present, such as amino, an acidic salt, i.e. hydrochloride, hydrobromide, acetate, palmoate, esylate, tosylate, and the like, can be used as the dosage form.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula (I) above.

The compounds of the invention may be in the form of pharmaceutically acceptable solvates. Pharmaceutically acceptable solvates of the compounds of Formula (I) and subformulae thereof contains stoichiometric or sub-stoichiometric amounts of one or more pharmaceutically acceptable solvent molecule such as ethanol or water. The term “hydrate” refers to when the said solvent is water.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

In another embodiment, the A2AR antagonist is an A2AR antagonist disclosed in WO2011/121418. Especially, the A2AR antagonist is the compound of example 1 of WO2011/121418, namely 5-bromo-2,6-di-(1H-pyrazol-1-yl)pyrimidin-4-amine, also known as NIR178:

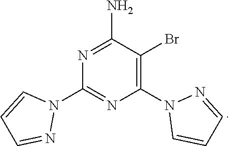 [SMILES: Nc1nc(-n2cccn2)nc(-n2cccn2)c1Br]

In another embodiment, the A2AR antagonist is an A2AR antagonist disclosed in WO2009/156737. Especially, the A2AR antagonist is the compound of example 1S of WO2009/156737, namely (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444:

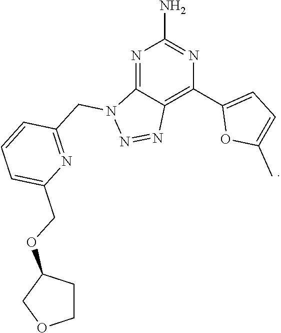 [SMILES: Cc1ccc(-c2nc(N)nc3c2nnn3Cc2cccc(CO[C@H]3CCOC3)n2)o1]

In another embodiment, the A2AR antagonist is an A2AR antagonist disclosed in WO2011/095626. Especially, the A2AR antagonist is the compound (cxiv) of WO2011/095626, namely 6-(2-chloro-6-methylpyridin-4-yl)-5-(4-fluorophenyl)-1,2,4-triazin-3-amine, also known as AZD4635:

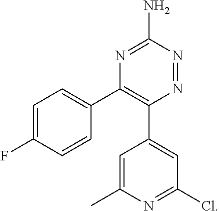 [SMILES: Cc1cc(-c2nnc(N)nc2-c2ccc(F)cc2)cc(Cl)n1]

In another embodiment, the A2AR antagonist is an A2AR antagonist disclosed in WO2018/136700. Especially, the A2AR antagonist is the compound of example 1 of WO2018/136700, namely 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928:

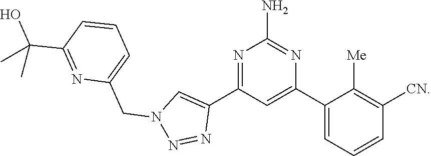 [SMILES: Cc1c(C#N)cccc1-c1cc(-c2cn(Cc3cccc(C(C)(C)O)n3)nn2)nc(N)n1]

In another embodiment, the A2AR antagonist is Preladenant (SCH-420,814), namely 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine:

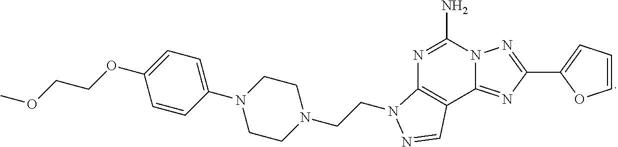 [SMILES: COCCOc1ccc(N2CCN(CCn3ncc4c3nc(N)n3nc(-c5ccco5)nc43)CC2)cc1]

In another embodiment, the A2AR antagonist is Vipadenant (BIIB-014), namely 3-(4-amino-3-methylbenzyl)-7-(2-furyl)-3H-(1,2,3)triazolo(4,5-d)pyrimidine-5-amine:

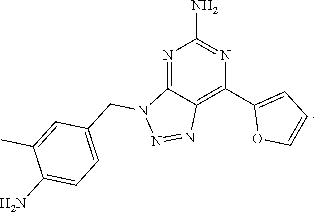 [SMILES: Cc1cc(Cn2nnc3c(-c4ccco4)nc(N)nc32)ccc1N]

In another embodiment, the A2AR antagonist is Tozadenant (SYK-115), namely 4-hydroxy-N-(4-methoxy-7-morpholinobenzo[d]thiazol-2-yl)-4-methylpiperidine-1-carboxamide:

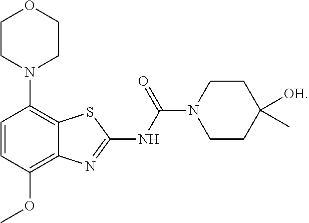 [SMILES: COc1ccc(N2CCOCC2)c2sc(NC(=O)N3CCC(C)(O)CC3)nc12]

Thus, in one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, the adenosine receptor antagonist is 5-bromo-2,6-di-(1H-pyrazol-1-yl)pyrimidin-4-amine. In one embodiment, the adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, the adenosine receptor antagonist is 6-(2-chloro-6-methylpyridin-4-yl)-5-(4-fluorophenyl)-1,2,4-triazin-3-amine. In one embodiment, the adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

### A2B Receptor Antagonist

In one embodiment, the combination of the invention includes at least one A2BR antagonist.

An “A2BR antagonist” refers to a compound that, upon administration to a patient, results in inhibition or down-regulation of a biological activity associated with activation of A2B receptor in the patient, including any of the downstream biological effects otherwise resulting from the binding to A2B receptor of its natural ligand. Such A2BR antagonists include any agent that can block activation of A2B receptor or any of the downstream biological effects of A2B receptor activation.

Examples of A2BR antagonists include: Vipadenant (BIIB-014), CVT-6883, MRS-1706, MRS-1754, PSB-603, PSB-0788, PSB-1115, OSIP-339,391, ATL-801, theophylline, Caffeine,

### Specific Combinations

In one embodiment, the combination of the invention comprises:
(a) an effective amount of an ENT inhibitor, preferably an ENT1 inhibitor, preferably selected from NBMPR, dipyridamole, dilazep, ticagrelor and salts thereof (including dilazep hydrochloride), and
(b) an effective amount of an adenosine receptor antagonist, preferably an A2AR antagonist, preferably selected from:
   (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   5-bromo-2,6-di-(1H-pyrazol-1-yl)pyrimidin-4-amine;
   (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
   6-(2-chloro-6-methylpyridin-4-yl)-5-(4-fluorophenyl)-1,2,4-triazin-3-amine;
   3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
   and pharmaceutically acceptable salts thereof.

In one embodiment, the combination of the invention comprises:
(a) an effective amount of dipyridamole as ENT inhibitor, and
(b) an effective amount of an adenosine receptor antagonist, preferably an A2AR antagonist, preferably selected from:
   (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   5-bromo-2,6-di-(1H-pyrazol-1-yl)pyrimidin-4-amine;
   (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
   6-(2-chloro-6-methylpyridin-4-yl)-5-(4-fluorophenyl)-1,2,4-triazin-3-amine;
   3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
   and pharmaceutically acceptable salts thereof.

In one embodiment, the combination of the invention comprises:
(a) an effective amount of an ENT inhibitor, preferably an ENT1 inhibitor, preferably selected from NBMPR, dipyridamole, dilazep, ticagrelor and salts thereof (including dilazep hydrochloride), and
(b) an effective amount of (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist.

In one embodiment, the combination of the invention comprises:
(a) an effective amount of an ENT inhibitor, preferably an ENT1 inhibitor, preferably selected from NBMPR, dipyridamole, dilazep, ticagrelor and salts thereof (including dilazep hydrochloride), and
(b) an effective amount of (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one as A2AR antagonist.

### Formulation and Pharmaceutical Composition

The invention further relates to formulation comprising the combination of the invention. Especially, the invention provides a formulation, comprising: an effective amount of an adenosine receptor antagonist in combination with an effective amount of an inhibitor of an ENT family member, along with a pharmaceutically acceptable excipient.

The specific embodiments relative to the adenosine receptor antagonists and inhibitors of an ENT family transporter recited above also apply in the context of the formulation of the invention.

The invention further relates to a pharmaceutical composition comprising the combination of the invention.

In one embodiment, the pharmaceutical composition comprises:
(a) an effective amount of an ENT inhibitor,
(b) an effective amount of an adenosine receptor antagonist; and
(c) at least one pharmaceutically acceptable carrier, diluent, excipient and/or adjuvant.

Above embodiments relative to the ENT inhibitors and adenosine receptor antagonists also apply to the pharmaceutical composition of the invention.

In a preferred embodiment, the invention provides a pharmaceutical composition comprising:
(a) an effective amount an ENT inhibitor, such as for example an ENT1 inhibitor;
(b) an effective amount an A2AR antagonist being a thiocarbamate derivative, more preferably a thiocarbamate derivative of Formula (I)

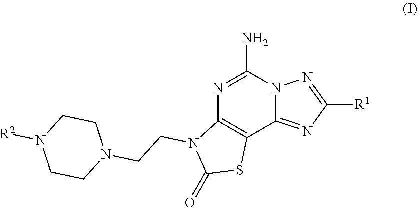

or a pharmaceutically acceptable salt or solvate thereof, as defined above; and
(c) at least one pharmaceutically acceptable carrier, diluent, excipient and/or adjuvant.

In one embodiment, the formulation or the pharmaceutical composition of the invention further comprises an additional therapeutic agent.

The at least one pharmaceutically acceptable carrier, diluent, excipient and/or adjuvant for use in the preparation of the administration forms will be clear to the skilled person; reference is made to the latest edition of Remington's Pharmaceutical Sciences.

Especially, the pharmaceutical composition of the invention can optionally contain such inactive substances that are commonly used in pharmaceutical formulations, such as for example cosolvents, lipid carrier, antioxidants, surfactants, wetting agents, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, and the like.

In a preferred embodiment, the pharmaceutical composition of the invention comprises one or more pharmaceutically acceptable cosolvent. Preferably cosolvents are selected from caprylic acid, polyethylene glycol (PEG), propylene glycol, ethanol, dimethylsulfoxide, dimethylacetamide, dimethylisosorbide and mixtures thereof. In a specific embodiment, the pharmaceutical composition of the invention comprises caprylic acid and/or PEG. Advantageously, when the composition comprises PEG as cosolvent, PEG is of low molecular weight, preferably PEG is PEG400. In an alternative embodiment, when the composition comprises PEG, it is of a moderate molecular weight, preferably PEG 3350.

In a specific embodiment, the pharmaceutical composition of the invention comprises one or more pharmaceutically acceptable lipid carrier. In a preferred embodiment, the lipid carrier is lauroyl polyoxyl-32 glycerides. This excipient corresponds to Gelucire® 44/14 manufactured by Gattefossé (Saint-Priest—France). This excipient is also known under the following references: lauroyl polyoxyl-32 glycerides NF/USP (NF: National Formulary; USP: US Pharmacopeia); lauroyl macrogol-32 glycerides EP (European Pharmacopeia); hydrogenated coconut PEG-32 esters (INCI); CAS number 57107-95-6. Gelucire® 44/14 corresponds to a well-defined multi-constituent substance constituted of mono-, di- and triglycerides and PEG-32 mono- and diesters of lauric acid (C₁₂). Gelucire® 44/14 has a melting point ranging from 42.5° C. to 47.5° C. (with a mean at 44° C.) and an hydrophilic/lipophilic balance (HLB) value of 14.

In another embodiment, the lipid carrier is Vitamin E TPGS. This excipient is also known under the following references: D-α-Tocopherol polyethylene glycol-1000 succinate; Tocophersolan; Tocofersolan; VEGS; α-[4-[[(2R)-3,4-dihydro-2,5,7,8-tetramethyl-2-[(4R,8R)-4,8,12-trimethyltridecyl]-2H-1-benzopyran-6-yl]oxy]-1,4-dioxobutyl]-ω-hydroxy-poly(oxy-1,2-ethanediyl); Vitamin E PEG succinate and is formed from Vitamin E which is conjugated to polyethylene glycol 1000 via a succinic acid linker. Vitamin E TPGS has melting point in the range 37-41° C. and an hydrophilic/lipophilic balance (HLB) value of 13.

In one embodiment, the pharmaceutical composition of the invention further comprises one or more antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

In some embodiments, surfactants are added, such as for example polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides), polylactide polyethylene glycol copolymer, polyvinyl caprolactam-polyvinyl acetate-polyethylene glycol graft copolymer (Soluplus®).

In some embodiments, wetting agents are added, such as for example sodium lauryl sulphate, vitamin E TPGS, sodium docusate, polysorbate 80, poloxamer 407. A preferred wetting agent is poloxamer 407.

In some embodiments, emulsifying agents are added, such as for example carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. Preferred emulsifying agents are for example poloxamer, propylene glycol monolaurate, propylene glycol dilaurate, and propylene glycol monocaprylate.

In some embodiments, buffering agents are used to help to maintain the pH in the range that approximates physiological conditions Suitable buffering agents include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

In some embodiments, pH modifiers are added, such as for example sodium hydroxide, sodium bicarbonate, magnesium oxide, potassium hydroxide, meglumine, sodium carbonate, citric acid, tartaric acid, ascorbic acid, fumaric acid, succinic acid and malic acid;

In some embodiments, preservatives agents are added to retard microbial growth. Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

In some embodiments, isotonifiers sometimes known as “stabilizers” are added and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; poloxamer 407; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. Preferred stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1, polyvinyl caprolactam-polyvinyl acetate-polyethylene glycol graft copolymer, polyvinylpyrrolidone polyvinylacetate copolymer.

In some embodiments granulating agent/binder(s) are added, such as for example starch, gums (inclusive of natural, semisynthetic and synthetic), microcrystalline cellulose, ethyl cellulose, methylcellulose, hydroxypropylcellulose, polymers such as povidone, polyvinylpyrrolidone polyvinylacetate copolymer and the like. Preferred granulating agents are for example methylcellulose, hydroxypropylcellulose, povidone and polyvinylpyrrolidone polyvinylacetate copolymer.

In some embodiments precipitation inhibitors are added, such as for example water soluble derivatives of cellulose including hydroxypropylmethylcellulose and methylcellulose, and water soluble polymers such as polyvinylpyrrolidone, polyvinylpyrrolidone polyvinylacetate copolymer, polyvinyl caprolactam-polyvinyl acetate-polyethylene glycol graft copolymer or poloxamer 407. A preferred precipitation inhibitor is hydroxypropylmethylcellulose.

In some embodiments lubricants are added, such as for example magnesium stearate, glyceryl esters, behenoyl polyoxyl-8 glycerides Nf (Compritol HD5 ATO), sodium stearyl fumarate and the like.

In some embodiments disintegrants are added, such as for example synthetics like sodium starch glycolate, cross povidone, cross carmellose sodium, kollidon CL, and natural origin such as locust bean gum and the like.

In some embodiments glidants are added, such as for example talc, magnesium stearate, colloidal silicon dioxide, starch and the like.

In some embodiments diluents (or fillers) are added, such as for example dextrose, lactose, mannitol, microcrystalline cellulose, sorbitol, sucrose, dibasic calcium phosphate, calcium sulphate dehydrate, starch and the like.

In some embodiments adsorbents are added, such as for example silicon dioxide, purified aluminium silicate and the like.

In some embodiments, the pharmaceutical composition of the invention is in the form of tablets and tableting excipients are added, such as for example granulating agents, binders, lubricants, disintegrants, glidants, diluents, adsorbents and the like.

In some embodiments the pharmaceutical composition of the invention is in the form of capsules, in which the capsule shells are constructed from gelatin or from non-animal derived products such as cellulose and its derivatives such as hydroxypropylmethylcellulose. Other ingredients may be included in the capsule shells such as polyethyleneglycol to act as plasticizer; pigments such as titanium dioxide or iron oxide to provide opacity and colour differentiation; lubricants such as carnauba wax; gelling agents such as carrageenan and wetting agents such as sodium lauryl sulphate. In one embodiment, the pharmaceutical composition of the invention is formulated as capsules, wherein the capsule shells are constructed from gelatin and wherein additional components are optionally included in the capsule shells, such as for example polyethylene glycol and sodium lauryl sulphate.

By means of non-limiting examples, the pharmaceutical composition may be in a form suitable for oral administration, for parenteral administration (such as by intravenous, intramuscular or subcutaneous injection or intravenous infusion), for topical administration (including ocular), for rectal administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

The compositions may be formulated so as to provide rapid, sustained or delayed release of the active compound(s) contained therein.

According to one embodiment, the pharmaceutical composition is in an adapted form for an oral administration. Forms adapted to oral administration may be solid, semi-solid or liquid. Some preferred, but non-limiting examples of such forms include liquid, paste or solid compositions, and more particularly tablets, tablets formulated for extended or sustained release, capsules (including soft and hard gelatin capsules), pills, dragees, lozenges, sachets, cachets, powder, liquids, gels, syrups, slurries, elixirs, emulsions, solutions, and suspensions.

According to another embodiment, the pharmaceutical composition is in an adapted form for an injection, especially to be injected to the subject by intravenous, intramuscular, intraperitoneal, intrapleural, subcutaneous, transdermal injection or infusion.

Sterile injectable forms of the pharmaceutical composition of the invention include sterile injectable solutions and sterile packaged powders (which are usually reconstituted prior to use) for administration as a bolus and/or for continuous administration.

Sterile injectable forms of the pharmaceutical composition of the invention may be a solution or an aqueous or oleaginous suspension. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent.

Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to another embodiment, the pharmaceutical composition of the invention is in an adapted form for a topical administration. Examples of forms adapted for topical administration include, without being limited to, liquid, paste or solid compositions, and more particularly aqueous solutions, drops, dispersions, sprays, ointments, cremes, lotions, microcapsules, micro- or nanoparticles, polymeric patch, or controlled-release patch, and the like.

According to another embodiment, the pharmaceutical composition of the invention is in an adapted form for a rectal administration. Examples of forms adapted for rectal administration include, without being limited to, suppository, micro enemas, enemas, gel, rectal foam, cream, ointment, and the like.

According to another embodiment, the pharmaceutical composition of the invention is in an adapted form for an administration by inhalation. Examples of forms adapted for administration by inhalation include, without being limited to aerosols.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

### Kit of Parts

The invention further relates to a kit of parts comprising the combination of the invention.

In one embodiment, the kit of parts of the invention comprises:
(a) a first part comprising an effective amount of an ENT inhibitor; and
(b) a second part comprising an effective amount of an adenosine receptor antagonist.

Above embodiments relative to the ENT inhibitors and adenosine receptor antagonists also apply to the kit of parts of the invention.

In a preferred embodiment, the invention provides a kit of parts comprising:
(a) a first part comprising an effective amount an ENT inhibitor, such as for example an ENT1 inhibitor; and
(b) a second part comprising an effective amount an A2AR antagonist being a thiocarbamate derivative, more preferably a thiocarbamate derivative of Formula (I)

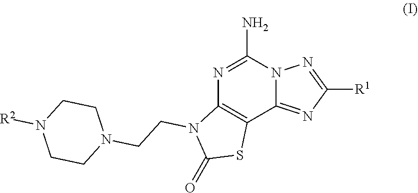

or a pharmaceutically acceptable salt or solvate thereof, as defined above.

Depending on the type of ENT inhibitor and adenosine receptor antagonist, the first and second parts of the kit may be under the form of pharmaceutical compositions. Excipients, dosage form and administration route of such pharmaceutical compositions will be clear to the skilled person (reference is made to the latest edition of Remington's Pharmaceutical Sciences), and especially may be those listed above with regards to the pharmaceutical composition of the invention.

In one embodiment, the second part of the kit comprises a pharmaceutical composition comprising an A2AR antagonist, preferably a thiocarbamate derivative of Formula (I) as defined above, and at least one pharmaceutically acceptable carrier, diluent, excipient and/or adjuvant. Pharmaceutically acceptable carrier, diluent, excipient and/or adjuvant of the pharmaceutical composition of the second part of the kit of part may be those listed above with regards to the pharmaceutical composition of the invention.

As developed below, the administration of the different parts of the kit may be made simultaneously or timely staggered, either at the same site of administration or at different sites of administration, under similar or different dosage forms.

In one embodiment, the kit of parts of the invention further comprises an additional therapeutic agent.

### Administration Regimen

In the context of the present invention, the administration of the ENT inhibitor and the adenosine receptor antagonist may occur either simultaneously or timely staggered, either at the same site of administration or at different sites of administration, under similar or different dosage forms as further outlined below.

In one embodiment, the ENT inhibitor is administered prior to, concomitant with, or subsequent to administration of an adenosine receptor antagonist.

To ensure that the separate mechanisms elicited by the ENT inhibitor and the adenosine receptor antagonist are not negatively influenced by each other, the adenosine receptor antagonist and the ENT inhibitor may be administered separated in time (in a time-staggered manner), i.e. sequentially, and/or are administered at different administration sites. This means that the adenosine receptor antagonist may be administrated e.g. prior, concurrent or subsequent to the ENT inhibitor, or vice versa. Alternatively or additionally, the adenosine receptor antagonist and the ENT inhibitor may be administered at different administration sites, or at the same administration site, preferably, when administered in a time staggered manner.

In one embodiment, the adenosine receptor antagonist is to be administered prior to and/or concomitantly with an ENT inhibitor. In one embodiment, the adenosine receptor antagonist is to be administered prior to the day or on the same day that the ENT inhibitor is administered.

In another embodiment, the ENT inhibitor is to be administered prior to and/or concomitantly with an adenosine receptor antagonist. In one embodiment, the ENT inhibitor is to be administered prior to the day or on the same day that the adenosine receptor antagonist is administered.

In one embodiment, the adenosine receptor antagonist is to be administered prior to and/or concomitantly with an ENT inhibitor and continuously thereafter.

In another embodiment, the ENT inhibitor is to be administered prior to and/or concomitantly with an adenosine receptor antagonist and continuously thereafter.

### Dose

Depending on the condition to be prevented or treated and the form of administration, the ENT inhibitor and the adenosine receptor antagonist may be administered as a single daily dose, divided over one or more daily doses.

It will be understood that the total daily usage of adenosine receptor antagonist and ENT inhibitor will be decided by the attending physician within the scope of sound medical judgment. The specific dose for any particular subject will depend upon a variety of factors such as the cancer to be treated; the age, body weight, general health, sex and diet of the patient; and like factors well-known in the medical arts.

In one embodiment, the subject is a mammal, preferably a human, and the dose of adenosine receptor antagonist, preferably a therapeutically effective dose, is a dose ranging from about 0.01 mg per kilo body weight (mg/kg) to about 5 mg/kg, preferably about 0.08 mg/kg to about 3.3 mg/kg, more preferably about 0.15 mg/kg to about 1.7 mg/kg.

In one embodiment, the subject is a mammal, preferably a human, and the dose of adenosine receptor antagonist, preferably a therapeutically effective dose, is a dose ranging from about 0.01 mg per kilo body weight per day (mg/kg/day) to about 5 mg/kg/day, preferably about 0.08 mg/kg/day to about 3.3 mg/kg/day, more preferably about 0.15 mg/kg/day to about 1.7 mg/kg/day.

In one embodiment, the subject is a mammal, preferably a human, and the dose of adenosine receptor antagonist, preferably a therapeutically effective dose, is a dose ranging from about 1 mg to about 500 mg, preferably about 5 mg to about 200 mg, more preferably from about 10 mg to about 100 mg.

In one embodiment, the subject is a mammal, preferably a human, and the dose of adenosine receptor antagonist, preferably a therapeutically effective dose, is a dose ranging from about 1 mg to about 500 mg per administration, preferably about 5 mg to about 200 mg per administration, more preferably from about 10 mg to about 100 mg per administration.

In one embodiment, the subject is a mammal, preferably a human, and the dose of adenosine receptor antagonist, preferably a therapeutically effective dose, is a daily dose ranging from about 1 mg to about 500 mg, preferably about 5 mg to about 200 mg, more preferably from about 10 mg to about 100 mg.

In one embodiment, the subject is a mammal, preferably a human, and the dose of adenosine receptor antagonist, preferably a therapeutically effective dose, is a daily dose to be administered in one, two, three or more takes. In one embodiment, the subject is a mammal, preferably a human, and the dose of adenosine receptor antagonist, preferably a therapeutically effective dose, is a daily dose to be administered in one or two takes.

In one embodiment, the subject is a mammal, preferably a human, and the dose of ENT inhibitor, preferably a therapeutically effective dose, is a dose ranging from about 0.01 mg per kilo body weight (mg/kg) to about 5 mg/kg.

In one embodiment, the subject is a mammal, preferably a human, and the dose of ENT inhibitor, preferably a therapeutically effective dose, is a dose ranging from about 0.01 mg per kilo body weight per day (mg/kg/day) to about 5 mg/kg/day.

In one embodiment, the subject is a mammal, preferably a human, and the dose of ENT inhibitor, preferably a therapeutically effective dose, is a dose ranging from about 1 mg to about 500 mg.

In one embodiment, the subject is a mammal, preferably a human, and the dose of ENT inhibitor, preferably a therapeutically effective dose, is a dose ranging from about 1 mg to about 500 mg per administration.

In one embodiment, the subject is a mammal, preferably a human, and the dose of ENT inhibitor, preferably a therapeutically effective dose, is a daily dose ranging from about 1 mg to about 500 mg.

In one embodiment, the subject is a mammal, preferably a human, and the dose of ENT inhibitor, preferably a therapeutically effective dose, is a daily dose to be administered in one, two, three or more takes. In one embodiment, the subject is a mammal, preferably a human, and the dose of ENT inhibitor, preferably a therapeutically effective dose, is a daily dose to be administered in one or two takes.

### Uses

Another object of this invention is the use of the combination as a medicament, i.e. for medical use. Thus, in one embodiment, the invention provides the use of the combination of the invention for the manufacturing of a medicament. Especially, the invention provides the use of the pharmaceutical composition of the invention or the kit of the invention for the manufacturing of a medicament.

Especially, the invention provides the combination, the pharmaceutical composition or the kit of parts of the invention, for use in the treatment and/or prevention of cancer.

The invention further provides the use of the combination, pharmaceutical composition or kit of parts of the invention for the manufacture of a medicament for treating and/or preventing cancer.

The invention further provides a method of treating of cancer, which comprises administering to a mammal species in need thereof a therapeutically effective amount of the combination, pharmaceutical composition or kit of parts of the invention.

Especially, the invention provides a method of treating cancer, comprising: administering, to a patient in need thereof, a combination of an adenosine receptor antagonist and an inhibitor of an ENT family transporter. The specific embodiments relative to the adenosine receptor antagonists and inhibitors of an ENT family transporter recited above also applies in the context of the methods of treatment of the invention.

The invention also provides for a method for delaying in patient the onset of cancer comprising the administration of a pharmaceutically effective amount of the combination, pharmaceutical composition or kit of parts of the invention to a patient in need thereof.

Various cancers are known in the art. Cancers that can be treated using the methods of the invention include solid cancers and non-solid cancers, especially benign and malignant solid tumors and benign and malignant non-solid tumors. The cancer may be metastatic or non-metastatic. The cancer may be may be familial or sporadic.

In one embodiment, the cancer to be treated according to the present invention is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present invention is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In another specific embodiment, the cancer is selected from the group consisting of: leukemia and multiple myeloma.

Preferably, the patient is a warm-blooded animal, more preferably a human.

In one embodiment, the subject receiving the ENT family transporter inhibitor is treated with an additional therapeutic agent in combination with the inhibitor of the ENT family transporter, or has received the additional therapeutic agent within about fourteen days of administration of the inhibitor of the ENT family transporter. In one embodiment, the additional therapeutic agent comprises an adenosine receptor antagonist.

In one embodiment, the subject has previously received at least one prior therapeutic treatment, and has progressed subsequent to the administration of the at least one prior therapeutic treatment and prior to administration of the inhibitor of an ENT family transporter. In one embodiment, the prior therapeutic treatment is selected from the group consisting of chemotherapy, immunotherapy, radiation therapy, stem cell transplant, hormone therapy, and surgery.

The invention also relates to a pharmaceutical formulation for use in the treatment of a cancer, wherein the pharmaceutical formulation is administered to a human subject in an amount effective to treat the cancer, and wherein the formulation comprises:
(a) an inhibitor of an ENT family transporter; and
(b) optionally one or more of a pharmaceutically acceptable carrier, diluent, excipient and/or adjuvant.

In one embodiment, in the pharmaceutical formulation for use of the invention, the ENT family transporter is ENT1, and the inhibitor is selected from the group consisting of a small molecule, a nucleic acid, a peptide, and an antibody.

In one embodiment, in the pharmaceutical formulation for use of the invention, the pharmaceutical formulation further comprises an additional therapeutic agent. In one embodiment, the additional therapeutic agent comprises an adenosine receptor antagonist.

In one embodiment, in the pharmaceutical formulation for use of the invention, he subject has previously received at least one prior therapeutic treatment. In one embodiment, the prior therapeutic treatment is selected from the group consisting of chemotherapy, immunotherapy, radiation therapy, stem cell transplant, hormone therapy, and surgery.

In one embodiment, in the pharmaceutical formulation for use of the invention, the pharmaceutical formulation is administered prior to, concomitant with, or subsequent to administration of the additional therapeutic agent comprising an adenosine receptor antagonist.

The invention also provides a pharmaceutical formulation for use in the treatment of a cancer, wherein the pharmaceutical formulation is administered to a human subject in an amount effective to treat the cancer, and wherein the formulation comprises:
(a) an inhibitor of an ENT family transporter
(b) an adenosine receptor antagonist; and
(c) optionally one or more of a pharmaceutically acceptable carrier, diluent, excipient and/or adjuvant.

In one embodiment, in the pharmaceutical formulations for use of the invention comprising an adenosine receptor antagonist, the adenosine receptor antagonist comprises an A2A or A2B receptor antagonist. In one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In another embodiment, the adenosine receptor antagonist comprises a compound of formula (I) as previously defined.

The invention also provides a method of treating a cancer comprising administering a pharmaceutical formulation to a human subject in an amount effective to treat the cancer, wherein the formulation comprises:
(a) an inhibitor of an ENT family transporter; and
(b) optionally one or more of a pharmaceutically acceptable carrier, diluent, excipient and/or adjuvant.

In one embodiment, in the method of the invention, the ENT family transporter is ENT1, and the inhibitor is selected from the group consisting of a small molecule, a nucleic acid, a peptide, and an antibody.

In one embodiment, in the method of the invention, the pharmaceutical formulation further comprises an additional therapeutic agent. In one embodiment, the additional therapeutic agent comprises an adenosine receptor antagonist.

In one embodiment, in the method of the invention, the subject has previously received at least one prior therapeutic treatment. In one embodiment, the prior therapeutic treatment is selected from the group consisting of chemotherapy, immunotherapy, radiation therapy, stem cell transplant, hormone therapy, and surgery.

In one embodiment, in the method of the invention, the pharmaceutical formulation is administered prior to, concomitant with, or subsequent to administration of the additional therapeutic agent comprising an adenosine receptor antagonist.

The invention also provides a method of treating a cancer comprising administering a pharmaceutical formulation to a human subject in an amount effective to treat the cancer, wherein the formulation comprises:
(a) an inhibitor of an ENT family transporter
(b) an adenosine receptor antagonist; and
(c) optionally one or more of a pharmaceutically acceptable carrier, diluent, excipient and/or adjuvant.

In one embodiment, in the methods of the invention wherein the pharmaceutical formulation comprises an adenosine receptor antagonist, the adenosine receptor antagonist comprises an A2A or A2B receptor antagonist. In one embodiment, the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In another embodiment, the adenosine receptor antagonist comprises a compound of formula (I) as previously defined.

### BRIEF DESCRIPTION OF THE DRAWINGS

FIGS. 1A, 1B and 1C are graphs representing the expression of ENT1, ENT2 and ENT4, respectively, in human primary lymphocytes. FPKMs: Fragments Per Kilobase Million. Source: Bonnal R J P, Nature 2015.

FIGS. 2A and 2B are graphs representing viability of CD3+ T cells under different treatments and show the reversal of Adenosine/ATP-mediated reduction in T cell viability by ENT inhibitors. Human primary CD3+ T cells were stimulated with antiCD3/antiCD28-coated microbeads, and treated with 25 μM adenosine (FIG. 2A) or 200 μM ATP (FIG. 2B). T cell viability was analysed by flow cytometry after a 3 days incubation period. Mean+/−SD of biological replicates are shown. Representative data for 1 healthy donor are shown. Similar data were obtained in 2 healthy donors.

FIGS. 3A, 3B, 3C and 3D are graphs representing proliferation of CD4+ T cells and CD8+ T cells under different treatments and show rescue of Adenosine/ATP-mediated suppression of T cell proliferation by ENT inhibitors. Human primary CD3+ T cells were stimulated with antiCD3/antiCD28-coated microbeads, and treated with 25 μM adenosine (FIGS. 3A and 3C) or 200 μM ATP (FIGS. 3B and 3D). Proliferation of CD4+(FIGS. 3A and 3B) and CD8+(FIGS. 3C and 3D) T cells was analysed by flow cytometry after a 3 days incubation period. Mean+/−SD of biological replicates are shown. Representative data for 1 healthy donor are shown. Similar data were obtained in 2 healthy donors.

FIGS. 4A and 4B are graphs representing IL-2 secretion by CD3+ T cells under different treatments and show rescue of Adenosine/ATP-mediated suppression of T cell cytokine secretion by a combination treatment with ENT inhibitors and A_{2A}R antagonist Compound 8a. CD3+ T cells were stimulated with antiCD3/antiCD28-coated microbeads, and treated with 25 μM adenosine (FIG. 4A) or 200 μM ATP (FIG. 4B). IL-2 secretion in the supernatant was analysed by AlphaLISA after a 3 days incubation period. Mean+/−SD of biological replicates are shown. Representative data for 1 healthy donor are shown. Similar data were obtained in 2 healthy donors.

FIG. 5A shows the viability of CD3+ T cells as determined by staining with a fixable viability dye. FIG. 5B shows the proportion of proliferating CD8+ T cells as determined by CFSE dilution. FIG. 5C shows the concentration of TNFα present in the cell culture supernatants. Human primary CD3+ T cells were stimulated with antiCD3/antiCD28-coated microbeads, and treated with 100 μM ATP or control medium. Viability of CD3+ T cells (FIG. 5A) and proliferation of CD8+ T cells (FIG. 5B) were analysed by flow cytometry after a 3 days incubation period. TNFα secretion in the supernatant was analysed by AlphaLISA. Grey shaded bars show A2AR antagonist treated wells, whilst clear bars denote concentration-matched DMSO treated wells. Results are shown as mean value from sextuplicate (DMSO) or duplicate (A2AR antagonists) wells±standard deviation. Data shown are representative of data obtained in three healthy individuals in 4 independent experiments.

FIG. 6 shows in vivo LPS induced TNFalpha production in mouse, upon prior treatment with Compound 8b (A2AR antagonist) or NBMPR (ENT1 inhibitor) or the combination thereof. BALB/c mice were pretreated for 1 h before LPS (1 mg/kg I.V) injection with NBMPR (20 mg/kg p.o.) or vehicle. Thirty minutes prior mice were treated with a single dose of Compound 8b (3 mg/kg p.o.) or vehicle. One hour after LPS treatment, blood serum was collected from the mice. TNFalpha levels were determined by ELISA (n=5). P values are indicated using Mann-Whitney test, ns=not significant.

FIG. 7 shows the MCA205 tumor growth upon treatment with Compound 8b (A2AR antagonist) used in combination with NBMPR (ENT1 inhibitor). FIG. 7A is a graph representing the median tumor volume over time after subcutaneous inoculation of tumor cells. FIG. 7B-D are the individual tumor growth volumes of mice treated with Vehicle (FIG. 7B) and NBMPR at 20 mg/kg BIDx22 (day 11) in standalone (FIG. 7C) or in combination with Compound 8b at 3 mg/kg BIDx22 (FIG. 7D). (n=10, *P=0.0043, **P=0.0015, ***P<0.0001, linear mixed model statistical analysis).

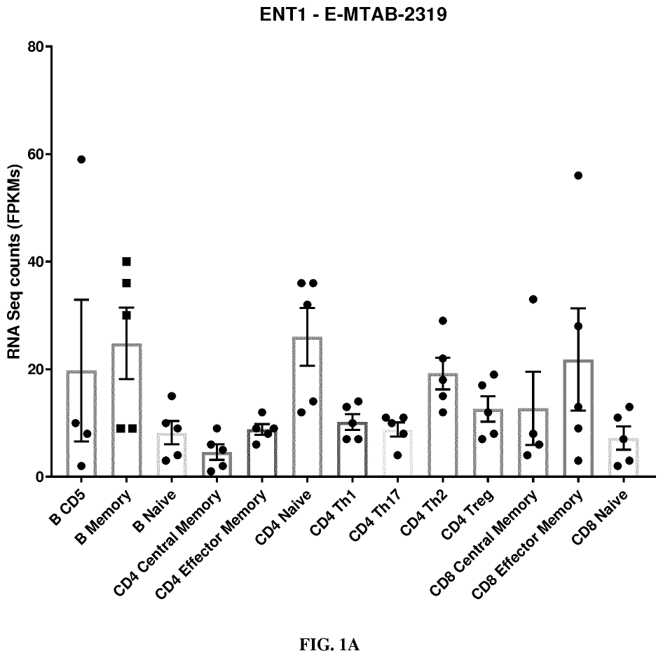

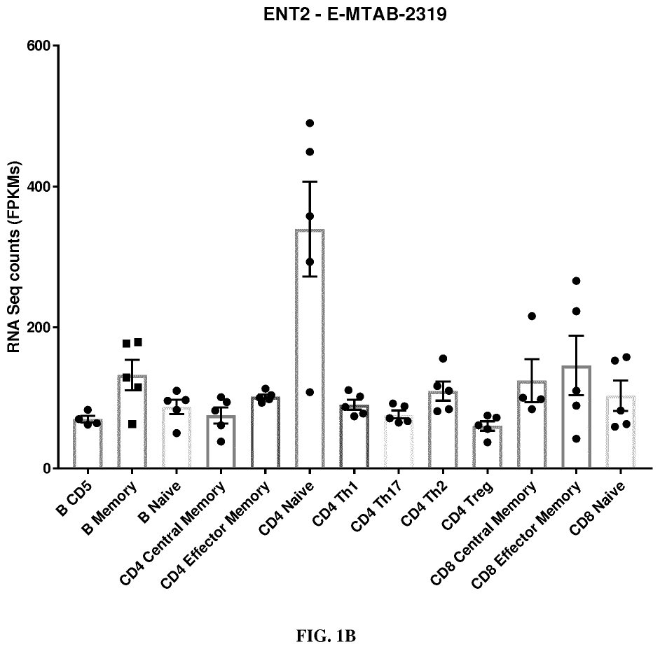

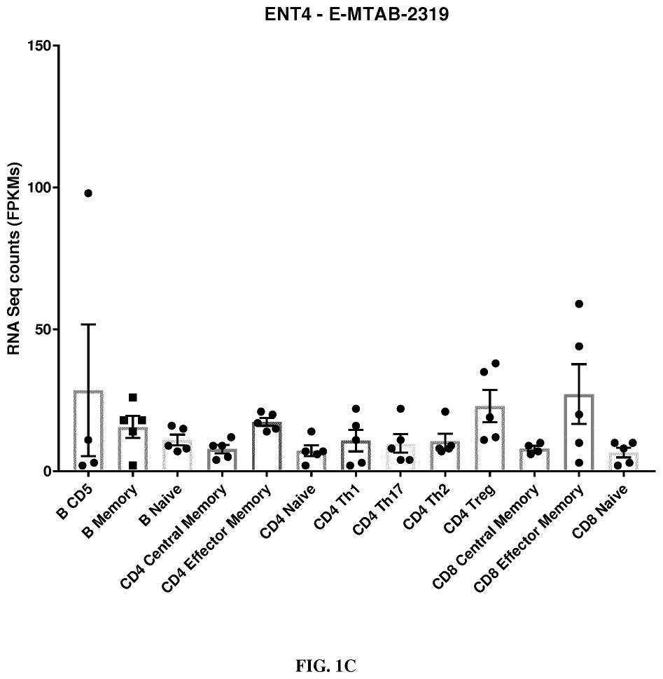

### EXAMPLES

The present invention will be better understood with reference to the following examples. These examples are intended to representative of specific embodiments of the invention, and are not intended as limiting the scope of the invention.

The following abbreviations are used:

ATP: adenosine triphosphate
BSA: bovine serum albumin
CFSE: carboxyfluorescein succinimidyl ester
DMSO: dimethylsulfoxide
EDTA: ethylenediaminetetraacetic acid
FACS: fluorescence-activated cell sorting,
FBS: fetal bovine serum
mL: milliliter
μL: microliter
mM: millimolar
nM: nanomolar
pM: micromolar
PBS: phosphate buffered saline
rpm: rounds per minute
RPMI: Roswell Park Memorial Institute medium

### Example 1. ENT1 Inhibitor in Combination with A2AR Antagonist Restores TNFalpha Production in T Cells

### Materials

Reagents and compounds used in the following assays have the following sources:

| Reagent | Source |
|---|---|
| SepMate-50 tubes | Stemcell Technologies |
| Lymphoprep | Elitechgroup |
| EasySep Human T Cell Isolation Kit | Stemcell Technologies |
| Fetal bovine serum (FBS) | Life Technologies |
| Dimethyl sulfoxide (DMSO) | Sigma-Aldrich |
| RPMI 1640 | Westburg (Lonza) |
| 1x non-essential amino acids | Westburg (Lonza) |
| Sodium Pyruvate | Westburg (Lonza) |
| Dynabeads human T-activator CD3/ | Life Technologies/ |
| CD28 | Thermo Fisher |
| X-Vivo 15 | Westburg (Lonza) |
| CFSE 1 | Life Technologies |
| PBS | Gibco/Lonza |
| BSA | Miltenyi biotec |
| EDTA | Westburg (Lonza) |
| Dipyridamole | Tocris |
| Dilazep dihydrochloride | Tocris |
| Ticagrelor | Sigma-Aldrich |
| Adenosine | Sigma-Aldrich |
| Adenosine 5′-triphosphate disodium salt | Sigma-Aldrich |
| hydrate (ATP) | |
| Compound 8a | prepared as described in |
| | PCT/EP2018/058301 |
| AlphaLISA Human IL2 Biotin Free | Promega |
| Detection kit | |
| AlphaLISA Human TNFα Biotin Free | Perkin-Elmer |
| Detection kit | |
| Anti-human CD3-APC | Biolegend (Imtec) |
| Anti-human CD4-BV786 | BD biosciences |
| Anti-human CD8-APC-Cy7 | Biolegend (Imtec) |
| Anti-human CD25-PE | Biolegend (Imtec) |
| Fixable Viability Dye eFluor506 | Life Technologies |
| CPI-444 | Prepared using a method adapted |
| | from patent WO2009/156737 |
| | (Example 1) |
| NIR178 | Prepared using a method adapted |
| | from patent WO2011/121418 |
| | (Example 1) |
| AZD4635 | Prepared using a method adapted |
| | from patent WO2011/095625 |
| | (compound number cxiv) |
| AB928 | Prepared using a method adapted |
| | from WO2019/161054 |
| | (Example 1) |

### Methods

PBMC and CD3+ T cell isolation. Venous blood from healthy volunteers, all of whom signed an informed consent approved by the Ethics Committee (FOR-UIC-BV-050-01-01 ICF_HBS_HD Version 5.0), was obtained via Unité d'Investigation Clinique (Centre Hospitalier Universitaire de Tivoli, La Louviere, Belgium). Mononuclear cells were collected by density gradient centrifugation, using SepMate-50 tubes and Lymphoprep according to the manufacturer's instructions. CD3+ T cells were isolated by immunomagnetic negative selection, using the EasySep Human T Cell Isolation Kit as per manufacturer's instructions. CD3+ T cells were stored in heat inactivated FBS and 10% DMSO in liquid nitrogen.

Human primary T cell culture. Human purified CD3+ T cells were thawed and washed twice with RPMI1640 medium, UltraGlutamine, supplemented with 1× non-essential amino acids (Lonza), and 1 mM Sodium Pyruvate (Gibco) (complete media), containing 10% hiFBS. After the final wash, cells were resuspended in PBS containing 10% FBS, and labeled using 3 μM CFSE during 5 min at room temperature, followed by two wash steps in complete media. After the last wash, cells were suspended at 1.6×10⁶ cells/mL in X-Vivol5 medium. 50 μL of cell suspension (8×10⁴ T cells) was added to wells of sterile round-bottom 96-well plates. Cells were activated by adding 50 μL of anti-CD3 anti-CD28 coated microbeads (Dynabeads human T-activator CD3/CD28), suspended in X-Vivo15 medium, at a ratio of one microbead per two cells. Adenosine (stock solution of 75 mM in DMSO) and ATP (powder) were diluted in X-Vivol5 medium, and 25 or 50 μL was added to the wells to reach final assay concentrations of 25 μM Adenosine or 200 μM ATP. Serial dilutions of the ENT inhibitors dilazep dihydrochloride, dipyridamole and ticagrelor were prepared in X-Vivol5 medium from 100 mM stock solutions (in DMSO or water), and 25 μL was added to the wells. The A_{2A}R antagonist Compound 8a (stock 10 mM in DMSO) was diluted in X-Vivol5 medium, and 25 μL was added to the wells to reach a final assay concentration of 300 nM. For studies including the different A2AR antagonists, wells received either the A2AR antagonist Compound 8a at a final concentration of 300 nM, NIR178 at a final concentration of 5 μM, CPI-444 at a final concentration of 5 μM, AZD4635 at a final concentration of 1 μM, or AB928 at a final concentration of 1 μM, or a matched concentration of DMSO. In addition, some wells received the ENT-1 inhibitor dipyridamole at a final concentration of 1 μM, or a combination of dipyridamole and A2AR antagonist. The final concentration of DMSO in the assay was 0.05%, and the final assay volume was 200 μL. Experiments were performed in biological duplicate, and DMSO control was added in quadruplicate. The cells were placed in a 37° C. humidified tissue culture incubator with 5% CO₂ for 72 hours. After 72 hours, the cells were pelleted by centrifugation at 1600 rpm for 5 minutes, and supernatants were transferred to V-bottom 96-well plates for cytokine quantification. Cell pellets were resuspended in FACS buffer (see below) for flow cytometry analysis.

Cytokine quantification. Supernatants were centrifuged at 4000 rpm for 10 minutes, and IL-2 was quantified using the IL-2 (human) AlphaLISA Biotin-Free Detection Kit, and TNFα was quantified using the AlphaLISA Human TNFα Biotin-Free Detection Kit, according to the manufacturer's instructions.

Flow cytometry. Cells were washed with FACS buffer (PBS containing 2 mM EDTA and 0.1% BSA) and stained with surface marker antibodies and viability dye for 20 min on ice. Cells were washed twice with FACS buffer and data acquired using an LSRFortessa (BD biosciences). Analysis was performed using FlowJo software (Treestar).

### Results

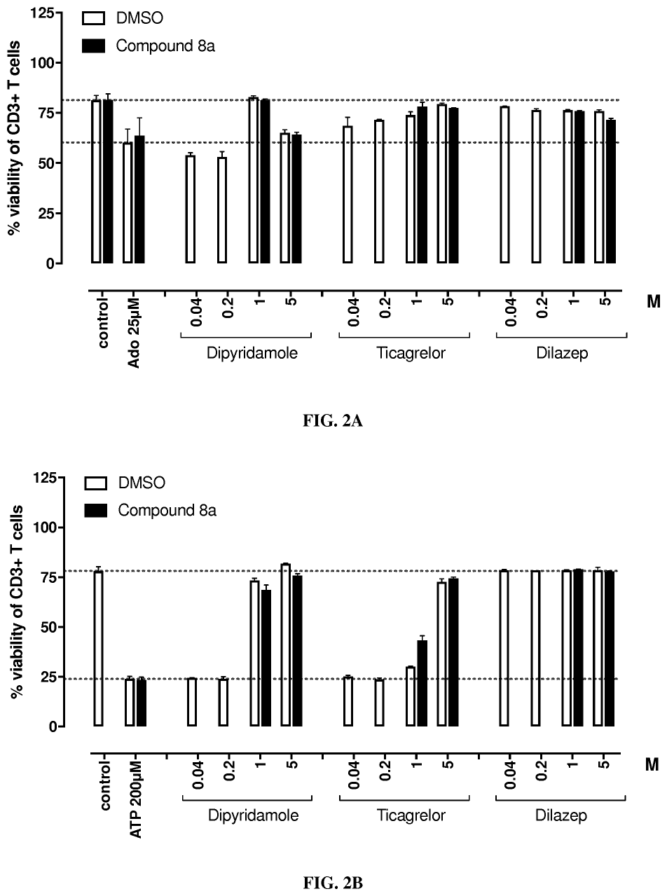

To evaluate effects of adenosine on human primary T cells, CD3+ T cells were stimulated with anti-CD3/CD28-coated microbeads, and treated with adenosine or ATP. Adenosine as well as ATP profoundly suppressed T cell proliferation and cytokine secretion (IL-2), and strongly reduced T cell viability (FIGS. 2-4).

Adenosine- and ATP-mediated suppression of T cell viability, proliferation and IL-2 secretion were not restored by Compound 8a, a potent and selective A2AR antagonist, suggesting an A2AR-independent mechanism of T cell suppression (FIGS. 2-4).

Extracellular adenosine is taken up by cells by Equilibrative Nucleoside Transporters (ENTs).

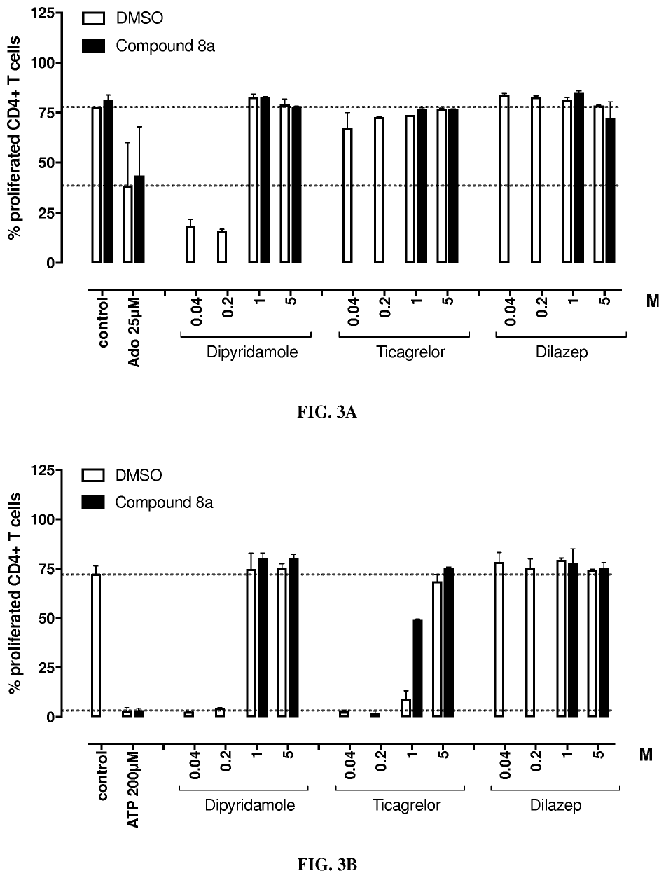

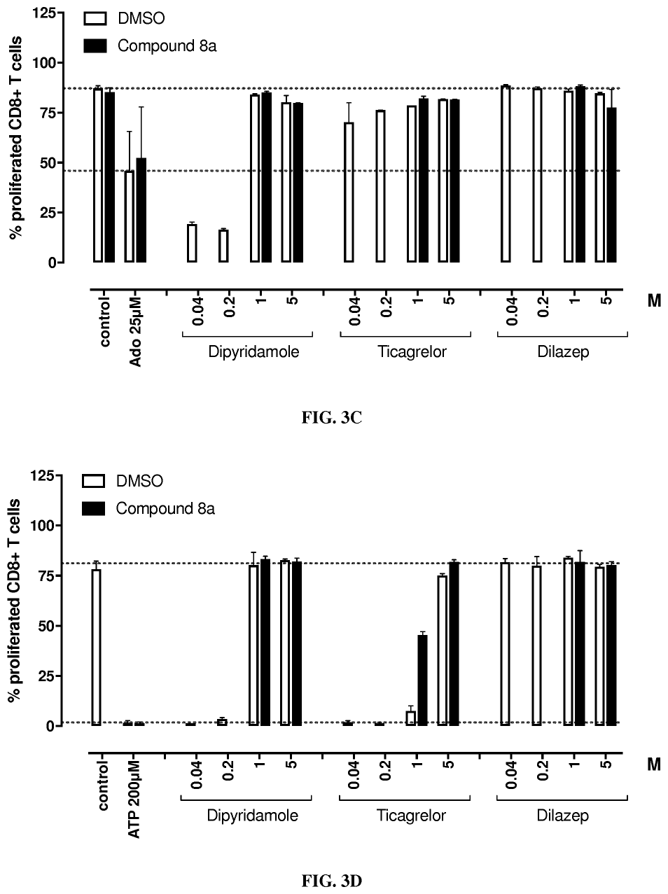

Primary T cells were treated with adenosine or ATP, and incubated with three different, structurally unrelated, ENT inhibitors: dipyridamole, dilazep hydrochloride and ticagrelor. All ENT inhibitors tested restored T cell proliferation and viability (FIGS. 2 and 3), thus illustrating the toxic effects of cellular uptake of adenosine by ENTs on T cell metabolism. However, none of the ENT inhibitors rescued T cell-derived IL-2 secretion as a single agent.

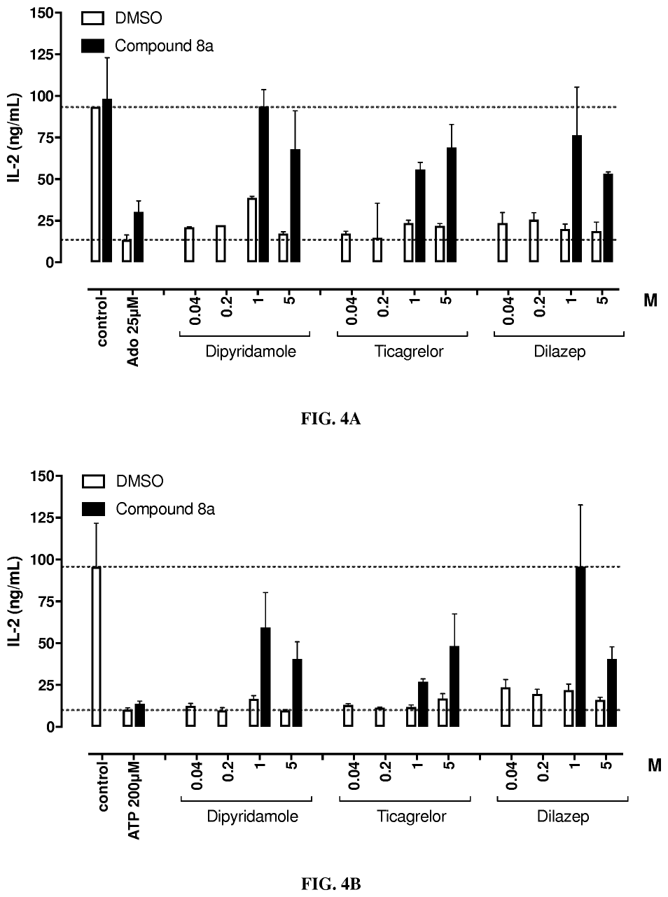

Complete rescue of adenosine- and ATP-mediated suppression of T cell cytokine secretion was achieved upon combined treatment with the A2A receptor antagonist Compound 8a and ENT inhibitors (FIGS. 4A and 4B). These results show that adenosine suppresses T cell function and vitality by both extracellular as well as intracellular mechanisms, which can be reversed by combined treatment with A2AR antagonists (not limited to Compound 8a—as shown further below) and ENT inhibitors.

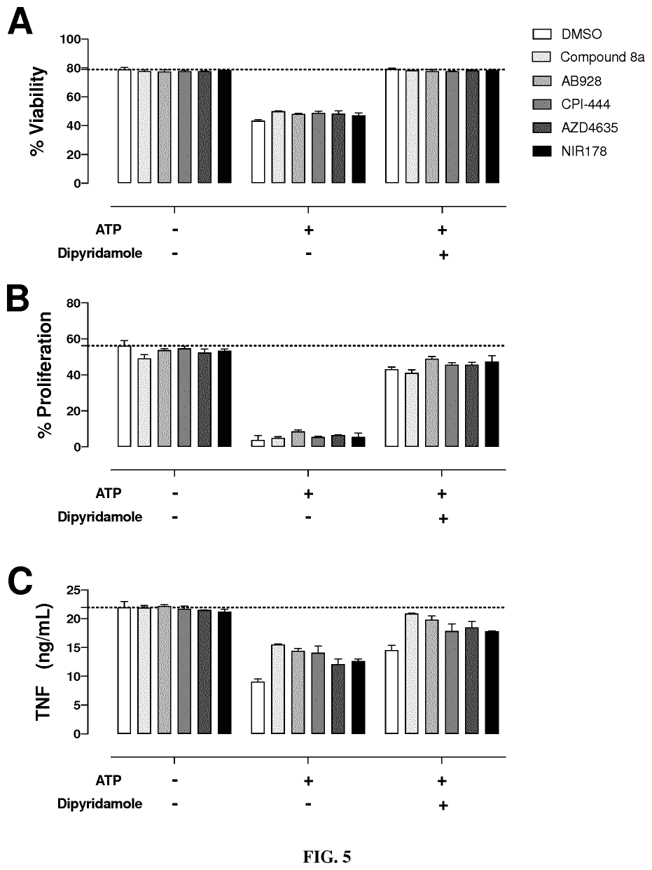

FIG. 5A-C illustrates that the inhibition of T cells in the presence of ATP may be reversed by a combination of the ENT-1 inhibitor dipyridamole and the A2A receptor antagonists Compound 8a, AB928, CPI-444, NIR178 and AZD4635. More specifically, ATP significantly inhibited CD8+ T cell proliferation and reduced T cell viability which was completely reversed by the ENT-1 inhibitor dipyridamole. ATP also significantly inhibited the production of TNFα. Both the A2AR and the ENT-1 inhibitors individually could partially restore cytokine production. The combination of both inhibitors further increased the restoration of cytokine secretion compared to either treatment alone. Overall, these data illustrate the value of combining Compound 8a, AB928, AZD4635, NIR178 or CPI-444 with an ENT-1 inhibitor for cancer therapy, aiming at full restoration of T cell function and viability in the ATP/adenosine-rich tumor microenvironment.

### Example 2. ENT1 Inhibitor in Combination with A2AR Antagonist Restores TNFalpha Production in an LPS Model of Endotoxemia

The most prominent target of adenosine transport is equilibrative nucleoside transporters, ENT1, 2, 3 and 4. ENT inhibitors regulate the import and export of nucleosides for example, adenosine across cell membranes such that inhibition of the equilibrative nucleoside transport may decrease the concentration of adenosine inside the cell and allow accumulation of adenosine extracellularly. High intracellular adenosine has been shown to lead to suppression of proliferation, survival and function of immune cells. On the other hand, extracellular adenosine by binding to the adenosine receptor 2A (A2AR) decreases activation and function, such as for example pro-inflammatory cytokine production and cytotoxicity of immune cells. Inhibition of equilibrative transporters therefore aims to decrease intracellular adenosine and thereby improving immune cell survival, proliferation and function, while A2AR inhibition aims to restore pro-inflammatory activity, cytotoxicity and immune cell activation by inhibiting extracellular adenosine.

NBMPR (6-S-[(4-Nitrophenyl)methyl]-6-thioinosine) is a specific ENT1 inhibitor and has similar activity in humans and mice. NBMPR is therefore used in mouse models to specifically inhibit the activity ENT1.

Using a Lipopolysaccharides (LPS) model, known to induce secretion of pro-inflammatory cytokine TNFalpha, it was assessed if TNFalpha-mediated suppression observed in presence of ENT1 inhibitor NBMPR, could be rescued by addition of A2AR antagonist, Compound 8b.

BALB/c mice were treated p.o. with Vehicle (2.5% DMSO, 10% Solutol HS15 in dH₂O pH3) as control or Compound 8b at 3 mg/kg. After 30 minutes, mice were treated with NBMPR p.o. at 20 mg/kg. 60 minutes later, mice were treated with LPS and blood was drawn for TNFalpha measurement in the serum, by ELISA.

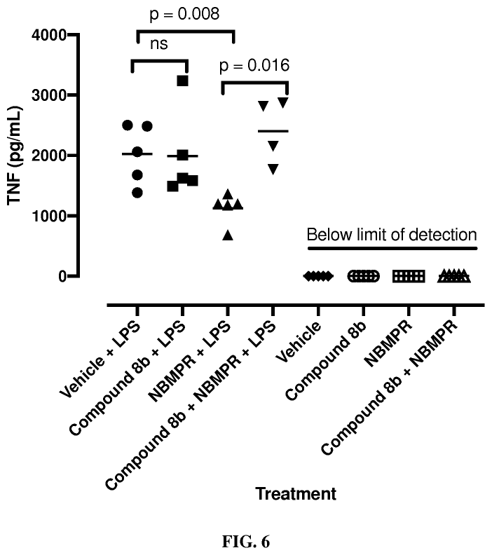

LPS treatment significantly elevated TNFalpha levels in the serum. A2AR antagonist, Compound 8b had no effect on TNFalpha levels after LPS treatment, while NBMPR treatment on its own, significantly suppressed TNFalpha production by almost 2 fold (p=0.008). Pretreatment of mice with Compound 8b, reversed the effects of NBMPR and rescued TNFalpha production by more than 2 fold (p=0.016) (FIG. 6).

### Example 3. ENT1 Inhibitor in Combination with A2AR Antagonist Demonstrates Antitumor Efficacy in Mouse Syngeneic MCA205 Experimental Fibrosarcoma Model

The anti-tumor efficacy of A2AR antagonist, Compound 8b, was assessed in combination with ENT1 inhibitor, NBMPR, in an established murine syngeneic MCA205 fibrosarcoma tumor model.

MCA205 tumor cells were inoculated subcutaneously into the right flank of C57BL/6 mice. When tumors reached an average size of about 50 mm, mice were randomly allocated into groups. Mice were administered vehicle p.o. (2.5% DMSO, 10% Solutol HS15 in dH₂O pH3) as control or NBMPR at 20 mg/kg p.o. BIDx22 (day7-29) as single agent or in combination with Compound 8b p.o. at 3 mg/kg QDx22 (day7-29).

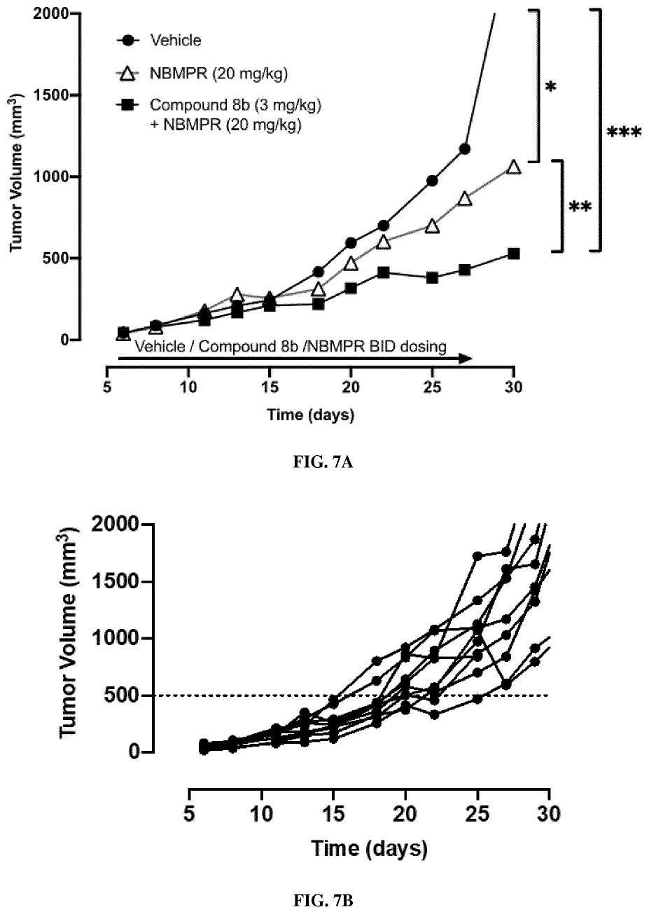

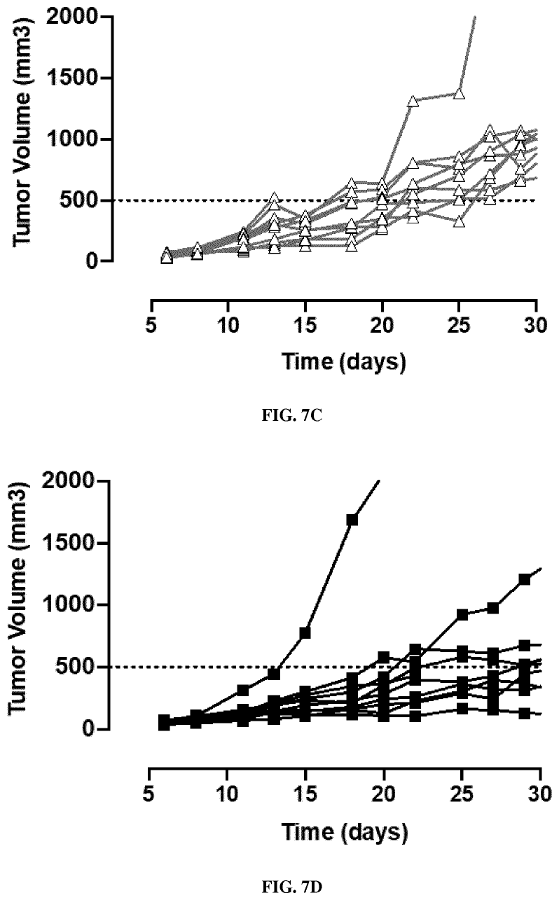

NBMPR, given as a single agent at 20 mg/kg p.o. twice daily (BID) for 22 consecutive days, demonstrated a significant delay in tumor growth (p=0.0043) compared to the Vehicle (FIGS. 7A, 7B and 7C).

Compound 8b, administered p.o. at 3 mg/kg in combination with NBMPR at 20 mg/kg, both twice daily (BID) for 22 consecutive days, demonstrated significant tumor growth delay compared to Vehicle (p<0.0001) and also significant tumor growth delay when compared to NBMPR single agent therapy (p=0.0015) (FIGS. 7A-7D).

1. An inhibitor of an ENT family transporter for use in the treatment of cancer in a human subject.

2. The inhibitor of an ENT family transporter for use according to claim 1, wherein the ENT family transporter is ENT1, and wherein the inhibitor is selected from the group consisting of a small molecule, a nucleic acid, a peptide, and an antibody.

3. The inhibitor of an ENT family transporter for use according to any one of claim 1 or claim 2, wherein the subject is treated with an additional therapeutic agent in combination with the inhibitor of the ENT family transporter, or has received the additional therapeutic agent within about fourteen days of administration of the inhibitor of the ENT family transporter.

4. The inhibitor of an ENT family transporter for use according to claim 3, wherein the additional therapeutic agent comprises an adenosine receptor antagonist.

5. The inhibitor of an ENT family transporter for use according to any one of claims 1-4, wherein the subject has previously received at least one prior therapeutic treatment, and has progressed subsequent to the administration of the at least one prior therapeutic treatment and prior to administration of the inhibitor of an ENT family transporter.

6. The inhibitor of an ENT family transporter for use according to claim 5, wherein the prior therapeutic treatment is selected from the group consisting of chemotherapy, immunotherapy, radiation therapy, stem cell transplant, hormone therapy, and surgery.

7. The inhibitor of an ENT family transporter for use according to claim 4, wherein the ENT family transporter inhibitor is administered prior to, concomitant with, or subsequent to administration of the additional therapeutic agent comprising an adenosine receptor antagonist.

8. A combination comprising
(a) an effective amount of an inhibitor of an ENT family transporter; and
(b) an effective amount of an adenosine receptor antagonist.

9. A pharmaceutical composition comprising:
(a) an effective amount of an inhibitor of an ENT family transporter;
(b) an effective amount of an adenosine receptor antagonist; and
(c) at least one pharmaceutically acceptable excipient.

10. A kit of parts comprising:
(a) a first part comprising an effective amount of an inhibitor of an ENT family transporter; and
(b) a second part comprising an effective amount of an adenosine receptor antagonist.

11. The combination, the pharmaceutical composition or the kit of parts according to any one of claim 8 to 10, wherein the adenosine receptor antagonist is an A2A or A2B receptor antagonist.

12. The combination, the pharmaceutical composition or the kit of parts according to any one of claim 8 to 11, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

13. The combination, the pharmaceutical composition or the kit of parts according to any one of claim 8 to 11, wherein the adenosine receptor antagonist is a compound of Formula (I): 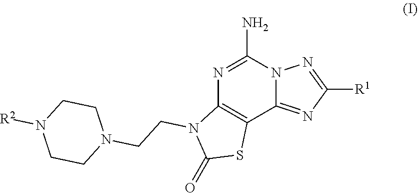
or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

14. The combination, the pharmaceutical composition or the kit of parts according to any one of claim 8 to 13, wherein the ENT family transporter is ENT1.

15. The combination, the pharmaceutical composition or the kit of parts according to any one of claim 8 to 14, further comprising an additional therapeutic agent.

16. The combination, the pharmaceutical composition or the kit of parts according to any one of claim 8 to 15, for medical use.

17. The combination, the pharmaceutical composition or the kit of parts according to any one of claim 8 to 15, for use in the treatment of cancer.

18. The combination, the pharmaceutical composition or the kit of parts according to any one of claim 8 to 15, wherein the ENT family transporter inhibitor is administered prior to, concomitant with, or subsequent to administration of an adenosine receptor antagonist.

19. A pharmaceutical formulation for use in the treatment of a cancer, wherein the pharmaceutical formulation is administered to a human subject in an amount effective to treat the cancer and wherein the formulation comprises:
(a) an inhibitor of an ENT family transporter; and
(b) optionally one or more of a pharmaceutically acceptable carrier, diluent, excipient and/or adjuvant.

20. The pharmaceutical formulation for use according to claim 19, wherein the ENT family transporter is ENT1, and wherein the inhibitor is selected from the group consisting of a small molecule, a nucleic acid, a peptide, and an antibody.

21. The pharmaceutical formulation for use according to any one of claim 19 or claim 20, wherein the pharmaceutical formulation further comprises an additional therapeutic agent.

22. The pharmaceutical formulation for use according to claim 21, wherein the additional therapeutic agent comprises an adenosine receptor antagonist.

23. The pharmaceutical formulation for use according to any one of claims 19 to 22, wherein the subject has previously received at least one prior therapeutic treatment.

24. The pharmaceutical formulation for use according to claim 23, wherein the prior therapeutic treatment is selected from the group consisting of chemotherapy, immunotherapy, radiation therapy, stem cell transplant, hormone therapy, and surgery.

25. The pharmaceutical formulation for use according to any one of claims 21 to 24, wherein the pharmaceutical formulation is administered prior to, concomitant with, or subsequent to administration of the additional therapeutic agent comprising an adenosine receptor antagonist.

26. A pharmaceutical formulation for use in the treatment of a cancer, wherein the pharmaceutical formulation is administered to a human subject in an amount effective to treat the cancer, and wherein the formulation comprises:
(a) an inhibitor of an ENT family transporter
(b) an adenosine receptor antagonist; and
(c) optionally one or more of a pharmaceutically acceptable carrier, diluent, excipient and/or adjuvant.

27. The pharmaceutical formulation for use according to any one of claims 22 to 26, wherein the adenosine receptor antagonist comprises an A2A or A2B receptor antagonist.

28. The pharmaceutical formulation for use according to any one of claims 22 to 27, wherein the adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

29. The pharmaceutical formulation for use according to any one of claims 22 to 27, wherein the adenosine receptor antagonist comprises a compound of formula (I) as defined in claim 13.
